(19) United States
(12) Patent Application Publication
(10) Pub. No.: US 2010/0075311 A1
(43) Pub. Date: Mar. 25, 2010

(54) CARTRIDGE SYSTEM

(75) Inventors: Denise Barrault, Midlothian (GB); Stuart Polwart, Midlothian (GB); David Thomson, Midlothian (GB); Jonathan Salmon, Midlothian (GB)

Correspondence Address: KNOBBE MARTENS OLSON & BEAR LLP, 2040 MAIN STREET, FOURTEENTH FLOOR, IRVINE, CA, 92614, US

(73) Assignee: ITI SCOTLAND LIMITED, GLASGOW (GB)

(21) Appl. No.: 12/443,070

(22) PCT Filed: Sep. 26, 2007
(86) PCT No.: PCT/GB07/03666

§ 371 Date: May 28, 2009

(30) Foreign Application Priority Data
Sep. 26, 2006 (GB) 0618966.6

(51) Int. Cl. C12Q 1/68; G01N 33/48; G01N 35/00; C12M 1/34; C12Q 1/00; G01N 33/00

(52) U.S. Cl. 435/6; 422/102; 422/68.1; 435/287.1; 435/287.2; 435/4; 436/86; 436/94

(57) ABSTRACT

A cartridge system includes a reagent component for storing one or more reagents and a processing component for processing the one or more reagents in an assay. The reagent component and the processing component are configured to be coupled together to form a cartridge. The reagent component and/or the processing component include at least one compartment configured to accept waste from the assay. The reagent component does not take part in processing the reagents in the assay, except to accept waste from the processing component. In one aspect, the cartridge system further includes a sensing component with at least one sensing element for detecting an analyte. In another aspect, the cartridge system further includes a sample preparation component for preparing a sample for the assay.

The present invention concerns a cartridge system for use in detecting one or more analytes in a sample, especially a biological sample. The system is typically a two-component system, and comprises a reagent component and a processing component. This is advantageous in that the reagent component can embody all of the assay specific elements (e.g. tailored to test for a specific medical condition) whilst the processing component can be a generic component compatible with a range of different types of sample, enabling a common processing instrument and reducing the cost of the users inventory. The invention also concerns a coupled cartridge, providing an environment in which the sample under test, all of the reagent components, and all of the waste reagents may be fully sealed and self contained within the cartridge assembly. This has the advantage of avoiding contamination and spillage risks and making disposal less hazardous. The system has particular utility in assays carried out in the near-patient environment, i.e. at the point of care (e.g. at a hospital clinic, a doctor's surgery or a patient's bedside). The present system is further advantageous in that its reagent component may comprise a compartment for accepting waste from the assay, thus simplifying cleaning and removing waste without the need for the user to come into contact with the waste. The invention also concerns methods for coupling the components, cartridges formed from the components, and assays performed using the components.

Conventional medical assays require one or more samples (such as blood or urine samples) to be taken from a patient in a hospital, or in a doctor's surgery, and then transferred to a laboratory for analysis. In the past, analysis of a sample in a “central” laboratory was unavoidable, due to the size and complexity of assay devices and systems. However, the requirement to analyse the sample in a remote location causes significant delay in diagnosing and treating a patient. In order to reduce the delay, there is an ongoing need to develop assay systems and methods that can be carried out in the near-patient environment, and that provide results quickly. Over time, smaller and less costly assay devices have been developed for this purpose.

It has been known for some time to employ cartridges in biological assay systems. Cartridges are advantageous in that they allow use of a single generalised assay device to assay for a number of different analytes by employing a different cartridge for each different analyte. They also simplify the assay procedure, in comparison with larger, more cumbersome laboratory systems. The development of microfluidic processing devices and chips has facilitated the development of such cartridges, since microfluidics allows much smaller (and cheaper) cartridges to be produced which can readily be inserted into a larger robust assay device. Published international application WO 02/090995 describes one such cartridge, which may be employed in a near-patient environment assay process.

However, there is still a need for the development of new cartridges and for the improvement of existing cartridges, to meet demand for new or more efficient assays, or assays capable of identifying several analytes simultaneously. Two-component cartridges have been developed in response to these needs. Typically, two-component cartridges have a component for storing reagents and a component for processing reagents with the sample. There are several advantages associated with these two-component systems. A separate reagent storage component simplifies the preparation and delivery of the solutions necessary for carrying out the assay. The component will be designed so as to maximise the shelf-life of the reagents and avoid the need for the user to control concentrations and volumes of solution. A two-component system provides more flexibility because a single processing component may be coupled with any one of a variety of reagent components, depending on the nature of the analyte under investigation. Published international application WO 2005/060432 describes a typical two-component cartridge for use with an electrochemical sensor. It describes systems which require both components to be specifically configured for a particular assay since the sensor component is integrated with the transport component, which generally requires a different configuration for, and is therefore specific to, each different assay to be performed.

Published patent, U.S. Pat. No. 4,940,527 discloses a two-part test cartridge for use in a centrifugal analyser. It is typically used for measuring the concentration of different electrolytes in blood. The cartridge contains a waste chamber and a sensor, the waste chamber being configured to accept excess sample, and to be disposable, whilst the sensor is on a re-usable portion of the cartridge.

Published patent application, US 2003/0073089 discloses a sensor cartridge for conducting chemical analysis, connected to a companion cartridge containing a reagent storage system and a waste retrieval system.

Further developments in cartridges are still required to improve efficiency and to simplify user operation, so that more complex assays can be carried out outside of a laboratory at the patient's point of care. It is an aim of the present invention to solve this problem, and the problems associated with known assay systems and cartridges, such as those described above.

Accordingly, the present invention provides a cartridge system comprising:
(a) a reagent component for storing one or more reagents; and
(b) a processing component for processing the one or more reagents in an assay;
   wherein the reagent component and the processing component are configured to be coupled together to form a cartridge, and wherein the reagent component and/or the processing component comprise at least one compartment configured to accept waste from the assay, the reagent component not taking part in processing the reagents in the assay, except to accept waste from the processing component.

The cartridge system of the present invention is particularly advantageous in that waste products from the assay may be neatly washed into a compartment, reservoir or void situated in the reagent component itself. This removes the need for the user to contact any waste products and conveniently seals them from the surroundings. This may be particularly important if any of the assay reagents are toxic, or if the sample under investigation is potentially infectious, or dangerous in any way. The system has the further advantage that the user does not need to handle or prepare any reagents, since they are stored in the reagent component. It is also advantageous in that the same processing component design may be employed for several different assays, simply by using different reagent components. It is also possible to readily design into the system different fluid paths for different analyses. In other words the system has increased flexibility due to its modularity and simplicity. The coupling of the two components eliminates or greatly reduces the risks of spillage and/or contamination. Moreover the liquid interfacing points (inlet and outlet ports which form the connection between the components when coupled together) are adaptable and may be anywhere on the interface (e.g. an interface plane) between the two components. The system is very safe and cost effective due to the disposability of the used cartridge. The system is also compact, and for example reagents and/or the sample may separated by membranes that can be broken by cartridge insertion into an assay device.

In order to achieve the advantages of the invention, the reagent component should not take part in processing the reagents in the assay, except to accept waste from the processing component. In known two-component cartridge systems, such as those in U.S. Pat. No. 4,940,527 and US 2003/0073089, the design of the two component system has been such that it has not been possible to eliminate all assay processing from the reagent storage component of the cartridge. In such a system the flexibility and simplicity are lost, since the design of the reagent component is not independent of the processing component. In the present invention, because the reagent component does not take part in processing the reagents in the assay, except to accept waste, the same reagent component design may be employed for a plurality of different processing components (i.e. a plurality of different assays). For each different assay, the reagents in the reagent component may differ, but the design of the reagent component voids and channels may remain constant.

The cartridge system typically comprises a sensing element for detecting an analyte (although in some embodiments the sensing element may be part of an assay device into which the cartridge is inserted and thus need not be present in the cartridge itself, or may be present in a third component (sensing component) of the system). The location of the sensing element or component is not especially limited, and may be selected depending upon the particular assay in question. Thus the sensing element or component may be part of the reagent component or the processing component. In a preferred embodiment the reagent component comprises the sensing element or component.

Thus the present invention also provides an embodiment in which a cartridge system comprises:
(a) a reagent component for storing one or more reagents;
(b) a processing component for processing one or more reagents in an assay; and
(c) a sensing component comprising at least one sensing element for detecting an analyte;
   wherein the reagent component, the processing component and the sensing component are separate components configured to be coupled together to form a cartridge.

In this embodiment, the sensor component is typically a separate third component of the cartridge, e.g. in a point-of-use kit. The sensor substrate can advantageously be pre-fabricated as a separate component prior to assembly in the reagent cartridge. This may include a method of applying probes to the surface of the sensor, such that having the sensor substrate as a separate discrete component is advantageous in the manufacture of the cartridge component e.g. in the case where probes are applied by an ink jet device operating close to the sensor surface when other features of the cartridge might inhibit this. The advantage of such an arrangement is, for example, that a variation in sensor substrate (e.g. probe density) may provide better specificity related to the estimated stage of a disease condition such as HCV.

It is preferred that the sensing component is configured to be coupled, optionally removably coupled, to either the reagent component or the processing component prior to coupling of the reagent and processing components. Typically, the sensing component and the reagent or processing component are provided pre-coupled to each other. In this context pre-coupled means that the sensing component and the reagent or processing component are separate components that are coupled together (optionally removably so) during manufacture, and are provided to the user (as part of a system or kit) in a coupled form along with a separate component (the other of the reagent or processing component that the sensor component is not coupled to). In all of these embodiments it is preferred that the reagent component comprises at least one compartment configured to accept waste from the processing component.

In a further embodiment, the invention provides a cartridge system comprising:
(a) a reagent component for storing one or more reagents;
(b) a processing component for processing one or more reagents in an assay; and
(c) a sample preparation component for preparing a sample for the assay;
   wherein the reagent component and the processing component are configured to be coupled together to form a cartridge.

In this embodiment, the system comprises a further component, a sample preparation component, which prepares the sample for the assay before delivering the prepared sample to the processing component. This embodiment offers many advantages. For example, different samples will require different types of preparation (e.g. a urine sample will be different from a blood sample) and the sample preparation component may allow such different samples to be used on the same processing component by pre-processing the sample before it is delivered to the processing component for carrying out the assay.

In all embodiments of the invention, it is also advantageous in that the cartridge can provide for simultaneous multi-analyte detection. A particularly advantageous means of achieving this is to configure the reaction chamber within which sensing takes place such that multiple methods of sensing can be employed, for example, both electro-chemical means and optical means.

In the context of the present invention all of the cartridge systems described may be in the form of a kit, for assembly and/or coupling at the point of use by a user.

To aid in this description, reference is made by way of example only to the following Figures, in which:

### Outline of the General Concept

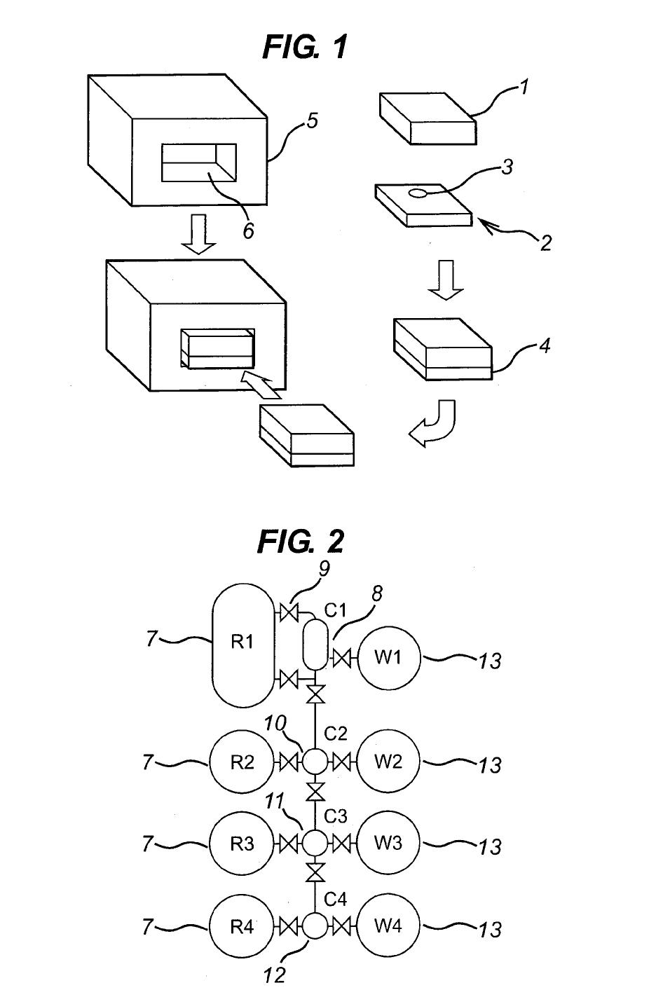

FIG. 1 illustrates the principle parts of the cartridge system—1 is a reagent storage component capable of storing multiple types of reagent in a variety of different volumes, 2 is a reagent processing component incorporating microfluidic channels, reaction zones and valving elements, 3 is a cavity for receiving a test sample, 4 is the complete processing cartridge which results from 1 and 2 being coupled together, 5 is the processing instrument which receives the cartridge through slot 6—the instrument 5 enables operation of various liquid transport, valving and detection means.

FIG. 2 is an example of some typical functional zones within the cartridge. 7 is a set of reagent storage chambers, each preferably containing a different reagent. For example, the chambers may independently contain running buffer, washing buffer, lysis buffer, and hybridisation buffer. 8 is a loading chamber which can receive a test sample which, for example, in its simplest form may be pre-purified nucleic acid extracts from a blood sample or in its most complex form, may be a whole blood sample. This loading can be achieved using a hand pipetting method, alternatively it can be achieved using an automated method inside the processing instrument. 9 is one of many valves embedded in the processing component, 10 and 11 are processing chambers within the reagent processing component within which, for example, cell lysis, washing steps or buffer exchange can take place. 12 is a reaction chamber within which the target analyte (for example, antibodies extracted from the test sample) is bound to probes on a sensor surface. Detection methods within the processing instrument, for example, fluorescent or luminescent imaging means, may be aligned with chamber 12 during the reaction sequence within chamber 12. 13 is a set of waste reagent storage chambers.

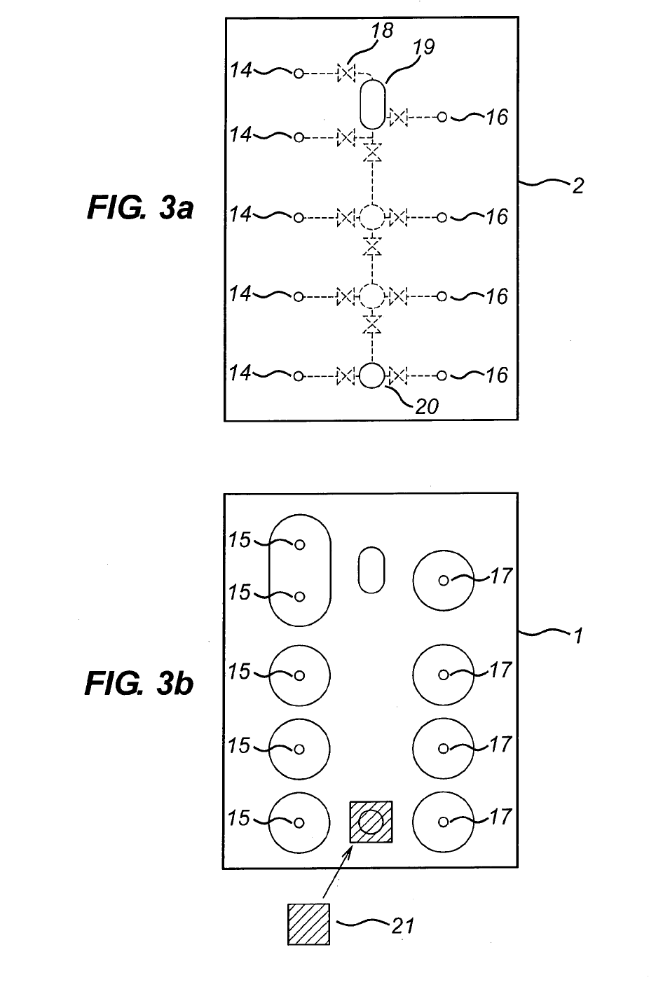

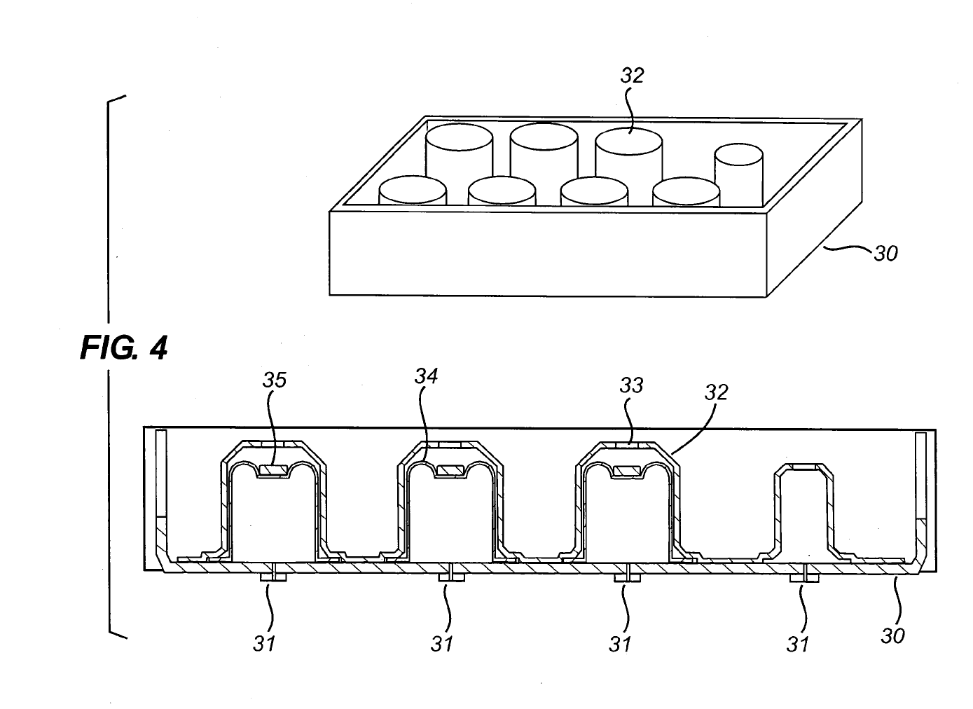

FIGS. 3a and 3b are corresponding examples of how these functions may conveniently be split between reagent storage component 1 and processing component 2. 14 is a set of liquid receiving ports to processing component 2. These ports 14 correspond to delivery ports 15 on the reagent storage component 1. 16 is a set of liquid delivery ports to reagent storage component 1. These ports 16 correspond to receiving ports 17 on the reagent storage component 1. 18 indicates channels and valves embedded below the top surface of the processing component 2. 19 is an open well for receiving the test sample. 20 is an open chamber which becomes a closed chamber when interfaced with substrate 21 on reagent storage component 1.

### Reagent Storage Component.

FIG. 4 shows an example where 30 is a plastic moulded carrier incorporating liquid port components 31 (which are embodiments of 15 and 17 in FIG. 3b), a storage housing 32 with external actuation apertures 33, liquid encapsulation membranes 34 and actuating pads 35. The reagent storage component optionally comprises a sub-component, which comprises typically a substrate to which various probes can be attached such as to provide a means of enabling interaction between the target analytes and those probes.

### Reagent Processing Component.

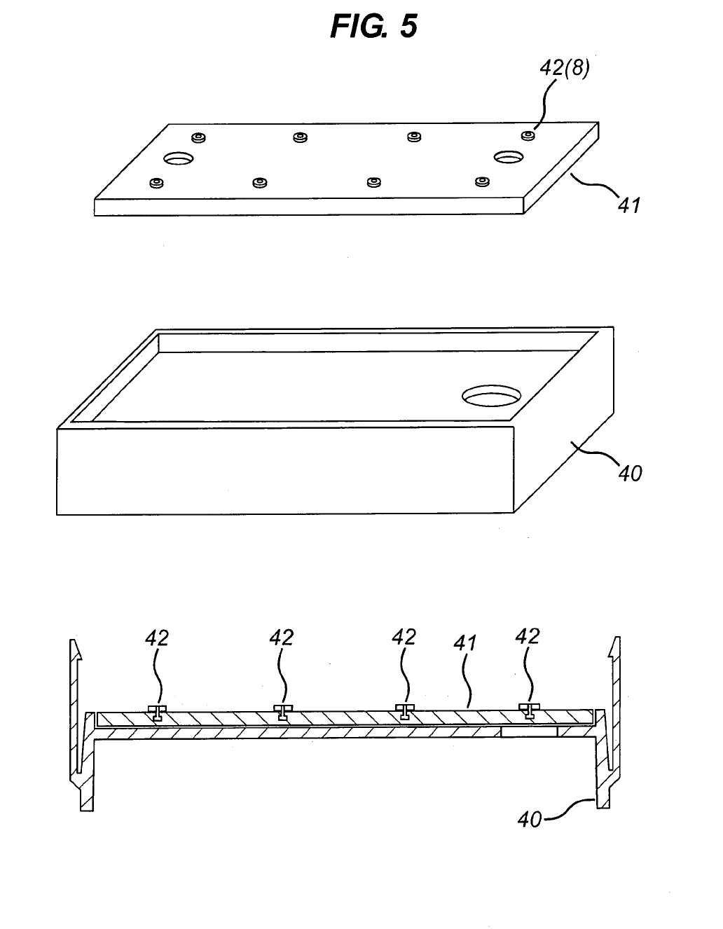

FIG. 5 shows an example where 40 is a plastic moulded carrier incorporating a microfluidic substrate 41 which in turn incorporates liquid port components 42 (which are embodiments of 14 and 16 in FIG. 3a). Possible internal arrangements of the microfluidic substrate 41 are known (for example, channel and cavity geometries, fabrication methods, valving methods, surface coatings) such that liquids may be transported, mixed, incubated and examined for analytical content. The present invention is capable of embodying many varieties of these prior art arrangements.

### Sensor Component

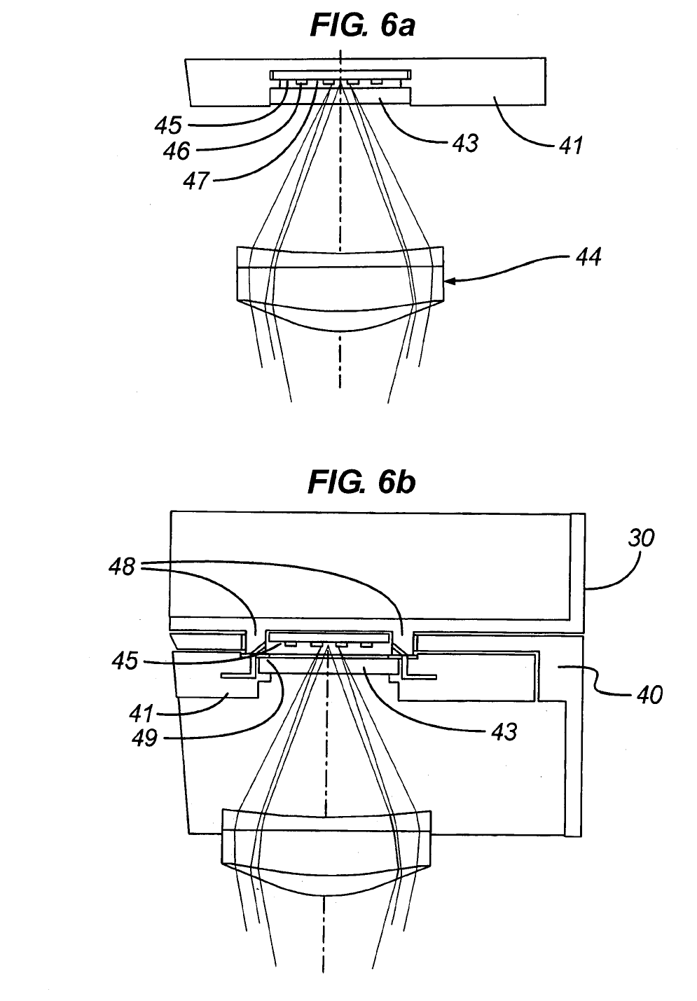

FIG. 6a shows an example where microfluidic substrate 41 incorporates on its underside a window 43 allowing lens system 44 to acquire an image of reaction processes on sensor substrate 45 which is attached to the upper zone of 41. This sensor substrate in combination with microfluidic substrate 41 creates a cavity 47 within which interaction between sensor probes 46 and a test analyte can take place.

FIG. 6b shows the same example but whereby the sensor substrate 45 is attached to carrier 30 of the reagent storage component. Ribs 48 provide registration and sealing against the face of window 43 via a compliant gasket 49 around the periphery of window 43 and these ribs may also incorporate channeling for transport of fluid in and out of the reaction chamber.

### Physical Coupling of the Cartridge Components

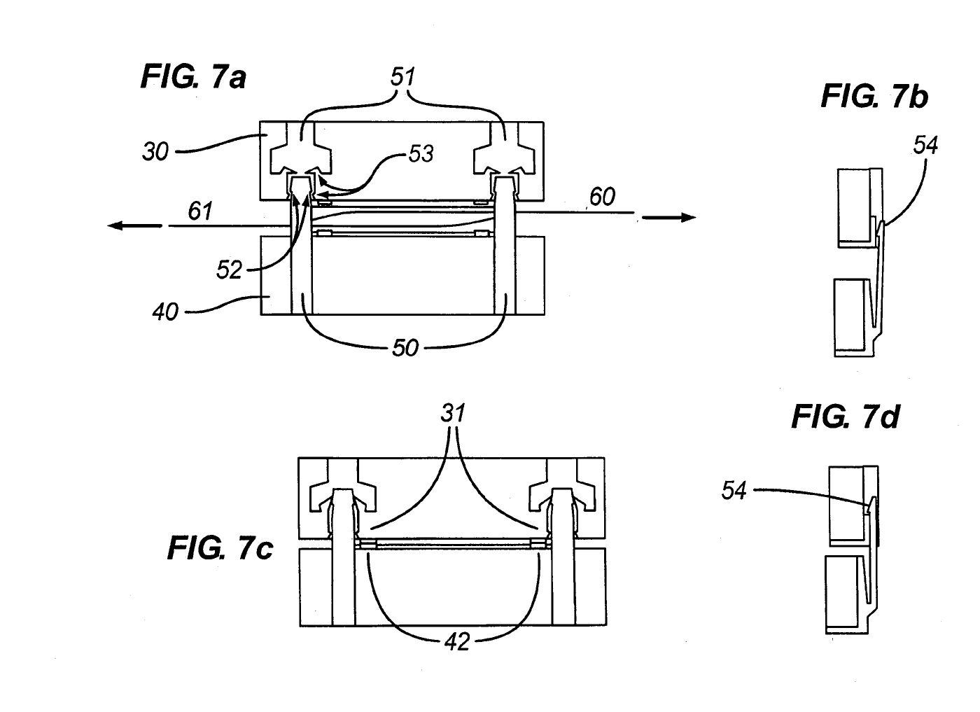

FIG. 7 shows an example where plastic moulded carrier 40 incorporates barbed plastic tongues 50 (two on each side of the cartridge) which engage with slots 51 in plastic moulded carrier 30 such that edge barbs 52 in combination with barbs 53 on plastic moulded carrier 30 provide a means of locating 30 and 40 together in two positions. The first position is a semi-engaged position shown in FIG. 7a which allows the end user to remove the protective guard strips 60 and 61 from carrier 30 and the optional substrate 45 (shown in FIG. 6b). The second position is shown in FIG. 7c and corresponds to a cartridge fully locked position whereby liquid ports 31 and 42 are fully engaged such that the test sample, the working reagents and the waste reagents are all fully contained within the cartridge. The locked position is enabled by a further barb 54 on the face of tongue 50. FIG. 7b shows this barb in the semi-engaged position and FIG. 7d shows this barb fully engaged by hooking over an edge on carrier 30. It is considered preferable that the transition from semi-engaged to fully engaged will be carried out automatically within the instrument after the user has loaded the cartridge. The instrument will embody a clamping mechanism to control this process. This action will result in bending of the upper barbs 53 as shown in FIG. 7c and this will result in a side clamping force to the tongues 50 thus ensuring very tight registration between the two cartridge components.

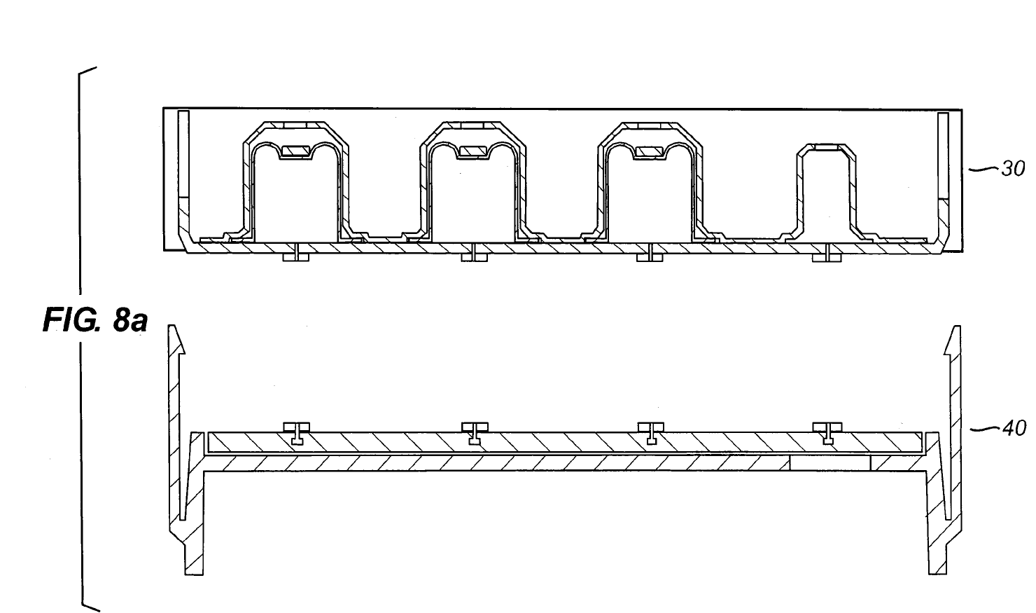

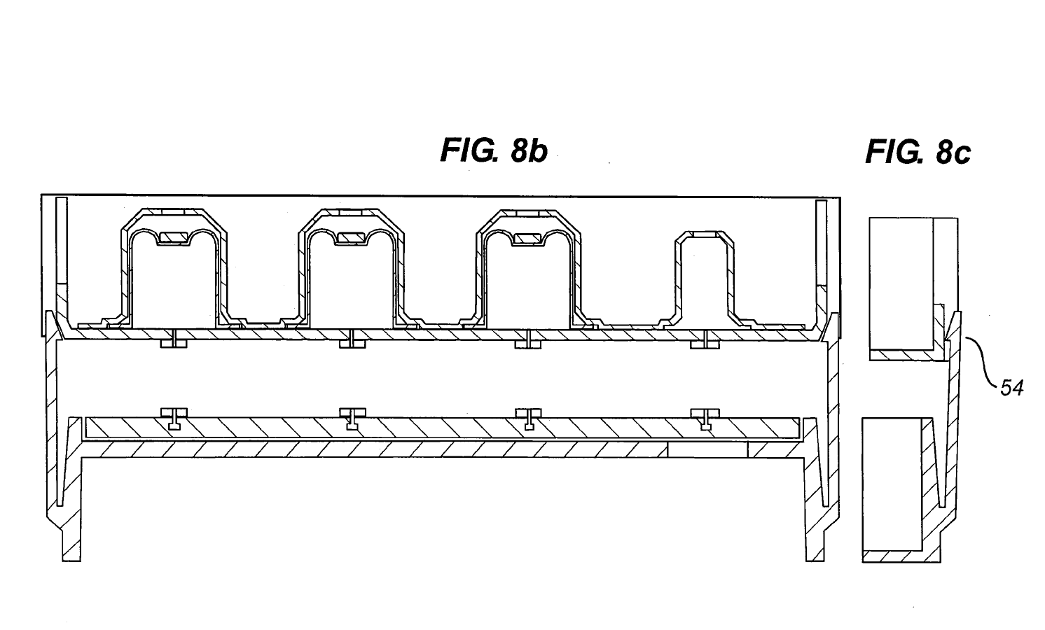

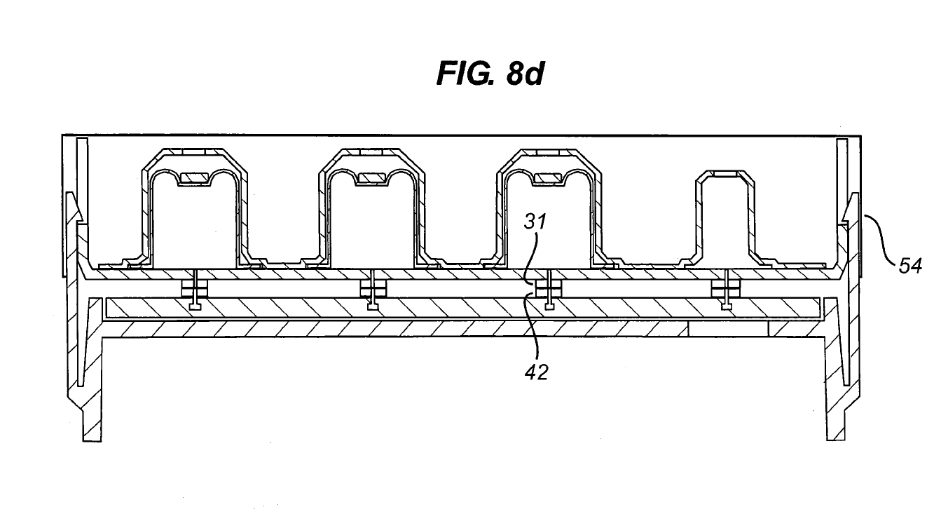

FIG. 8 shows an exemplary sequence of engagement between carrier 30 and carrier 40.

### Liquid Coupling Between the Cartridge Components

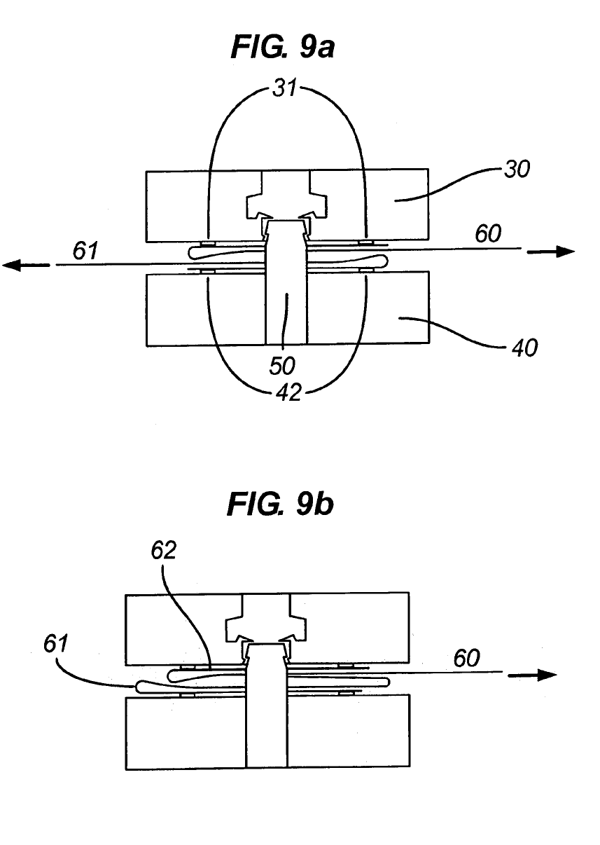

FIG. 9a shows an example of the use of protective guard strips 60 and 61. Strip 60 is, for example, heat sealed to carrier 30 at the point of reagent filling during the manufacturing process such that the reagents are sealed and isolated from the outside environment. Strip 60 can also provide the same protective function for the sensor substrate 45 (ref FIG. 6b) and strip 61 can also provide the same protective function for loading well 19 (ref FIG. 3b). FIG. 9a also shows an alternative tongue and barb arrangement whereby one central tongue is employed on either side of the cartridge. The position illustrated is the semi-engaged position where the two components are aligned and ready for removal of the protective strips. These protective strips may be removed by the user by pulling one strip from each side as in FIG. 9a. FIG. 9b shows that the strips can also be linked together by an adhesive attachment at 62 and in this manner, a single pull from one side will remove both strips.

### An Example Biological Assay

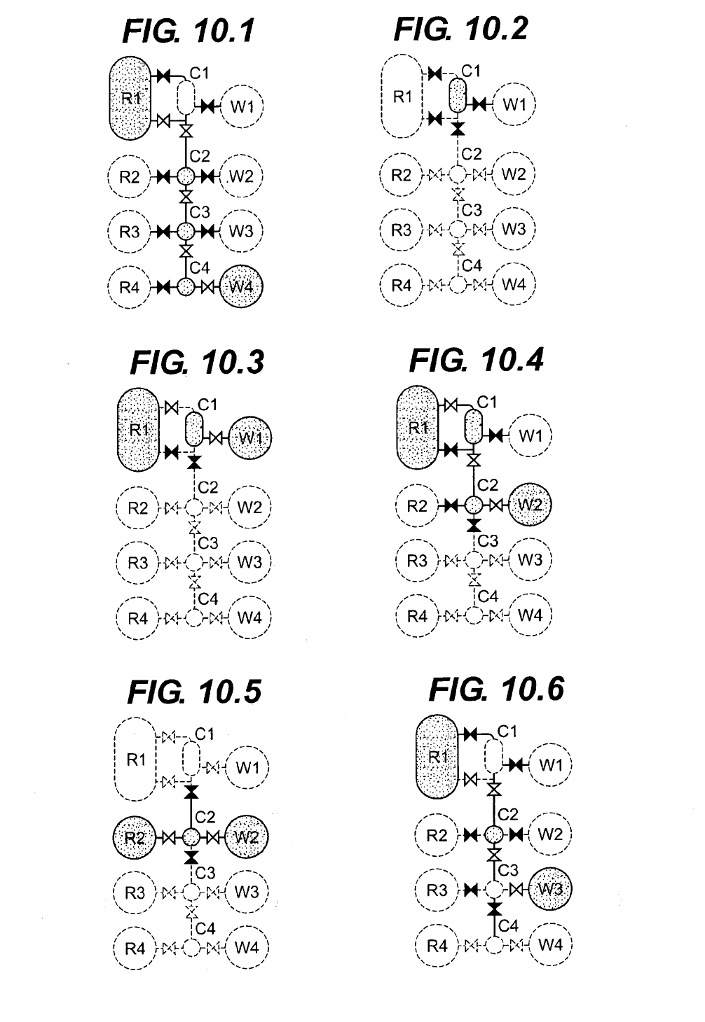

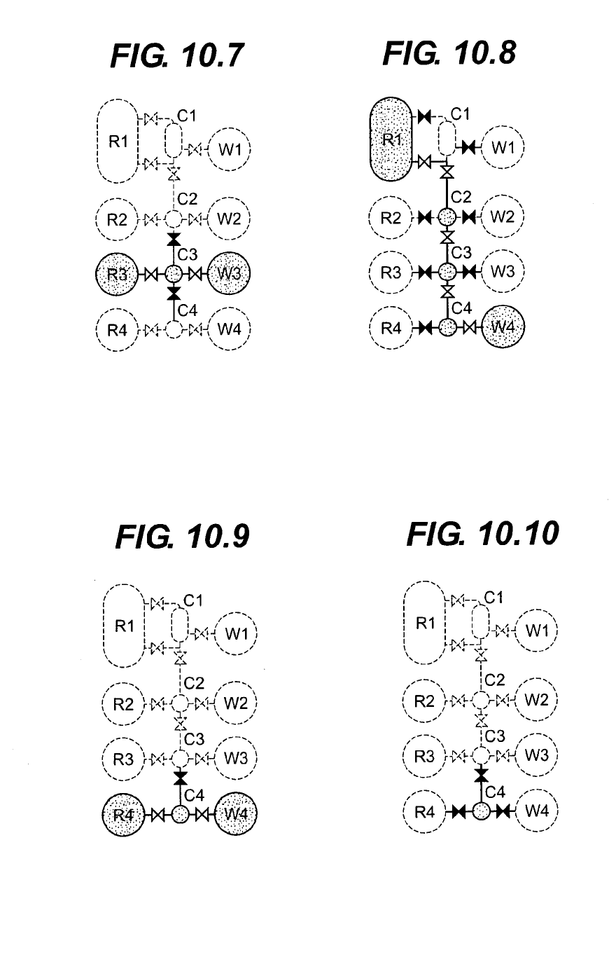

FIG. 10 shows a reagent flow sequence corresponding to that required for a simple ELISA type assay (see example 1 below).

### An Example Cartridge Interconnect

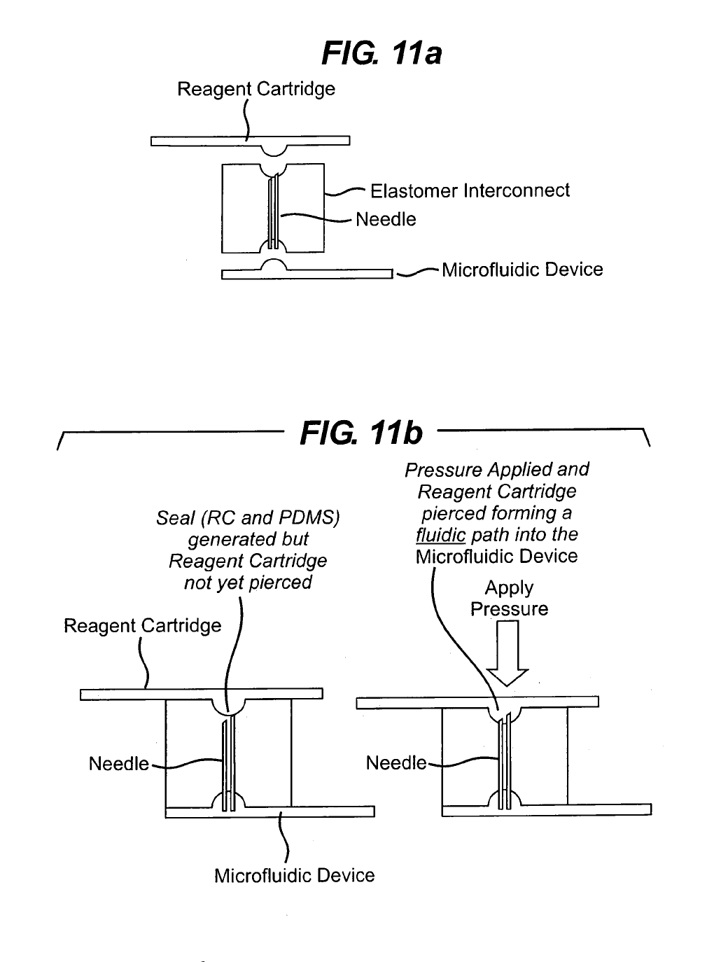

FIGS. 11a and 11b show an exemplary cartridge interconnect system.

### An Example of a Sample Zone

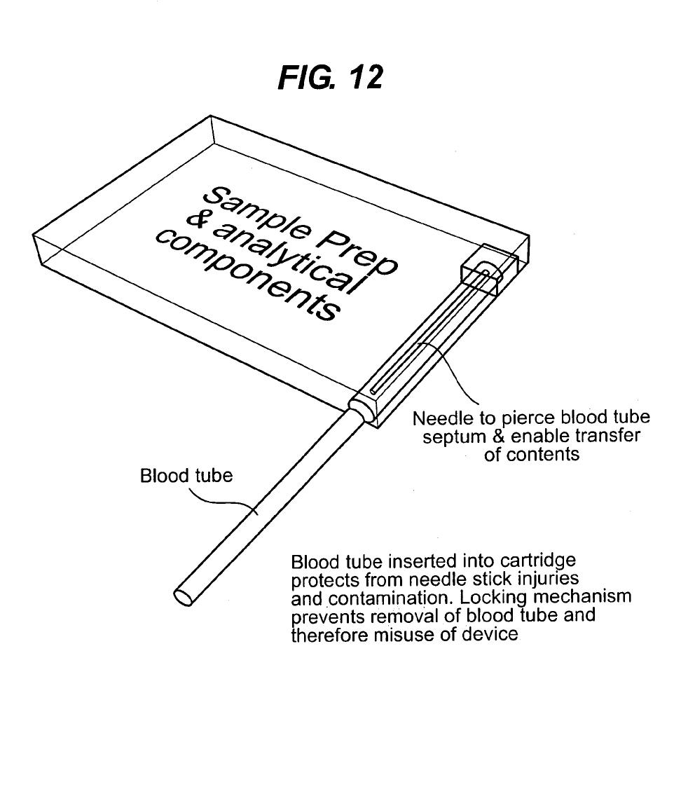

FIG. 12 shows a sample zone at the edge of a cartridge system. This sample zone is configured to accept a blood tube (i.e. the sample is a whole blood sample). It is advantageous since the needle for piercing the blood tube is hidden within the sample zone to protect the user from needle stick injuries, and contamination.

### Prototype Processing Component

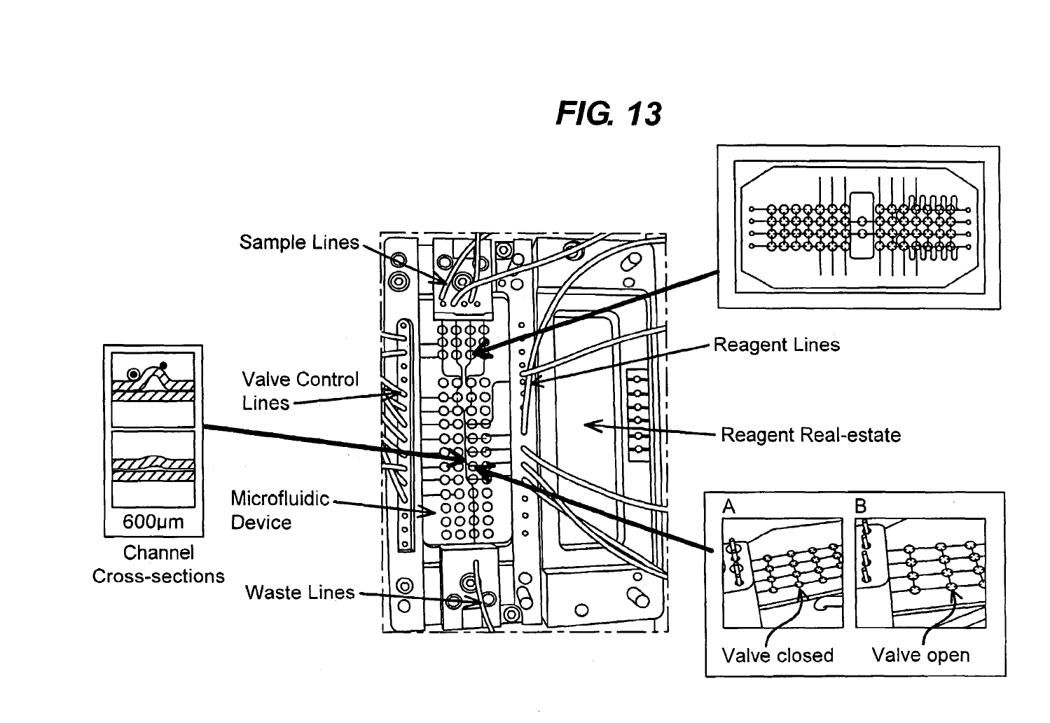

FIG. 13 shows a prototype of the processing component in development. The processing component is labeled as the microfluidic device, and the reagent lines and waste lines can cbe clearly seen. These lines are to be connected to the reagent storage component, seen on the right of the processing component. The Figure also shows the valving systems and the dimensions of the microfluidic channels.

### Example Layout for Processing Component

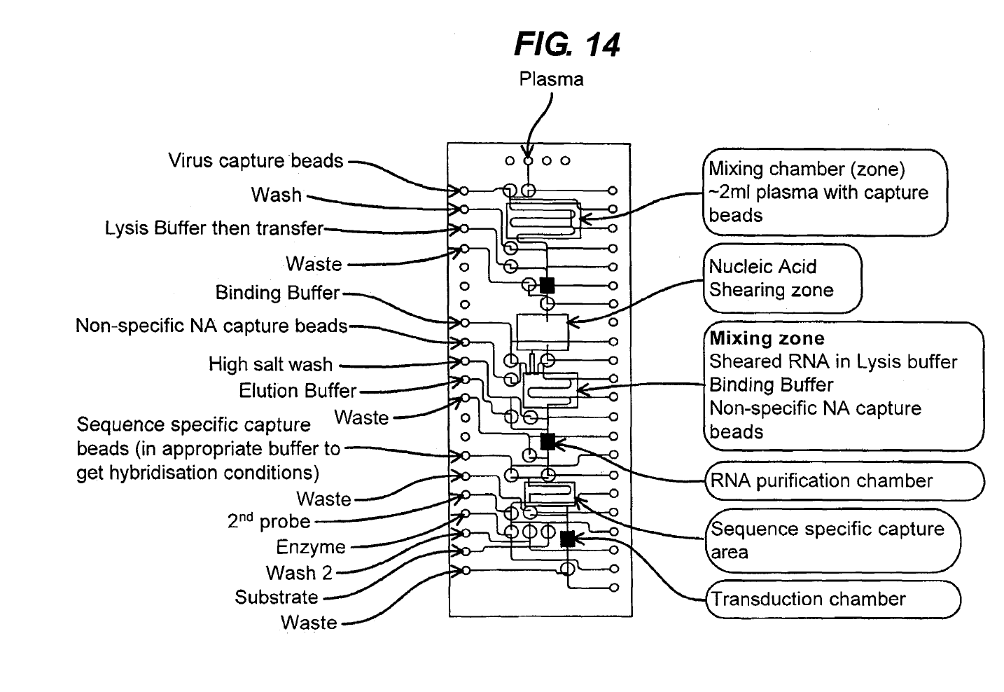

FIG. 14 shows an example of the layout of the processing component, in this case for a nucleic acid assay.

### Further Example Layout for Processing Component

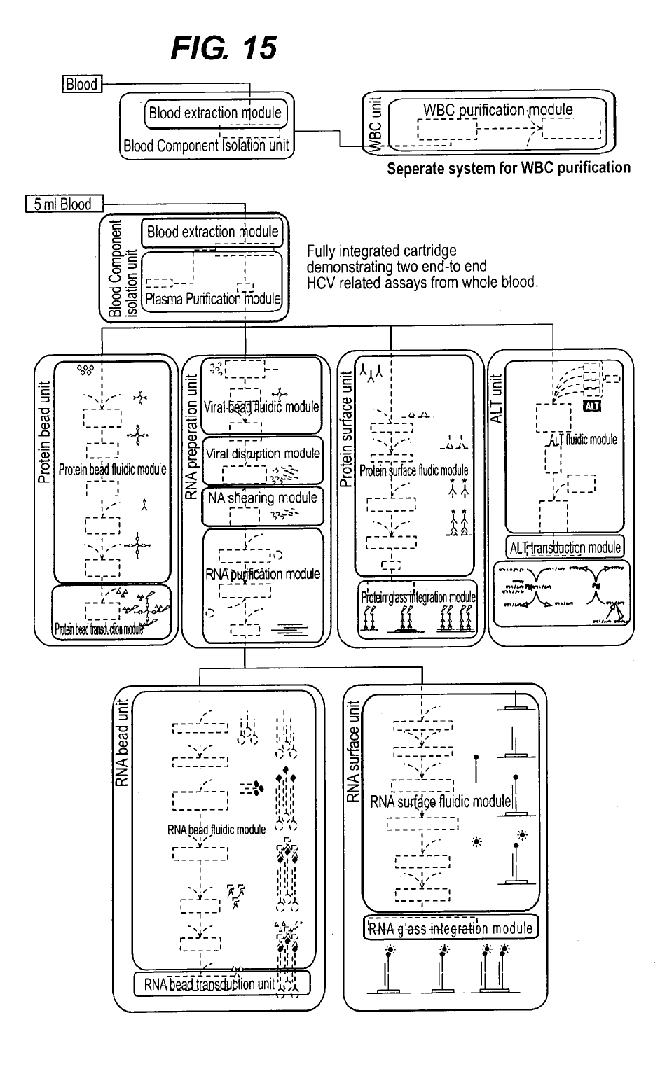

FIG. 15 shows an example of a number of possible processing components, in an HCV assay. The diagram shows two possible configurations for the sample preparation component (in this case a blood separation component)—a separate separation component (the top two boxes showing the extraction of white blood cells (WBC)) and an integrated component (second box from the top, showing a plasma purification module). This illustrates a general principle of the present invention that the sample preparation component may be coupled (or capable of coupling) to the other components, or may be separate. When the sample preparation unit is separate from the other components, the transfer of the prepared sample may nevertheless be automated in some way, for example through fluid lines connecting one unit from another separate unit.

### Example Layout of Sample Preparation Component

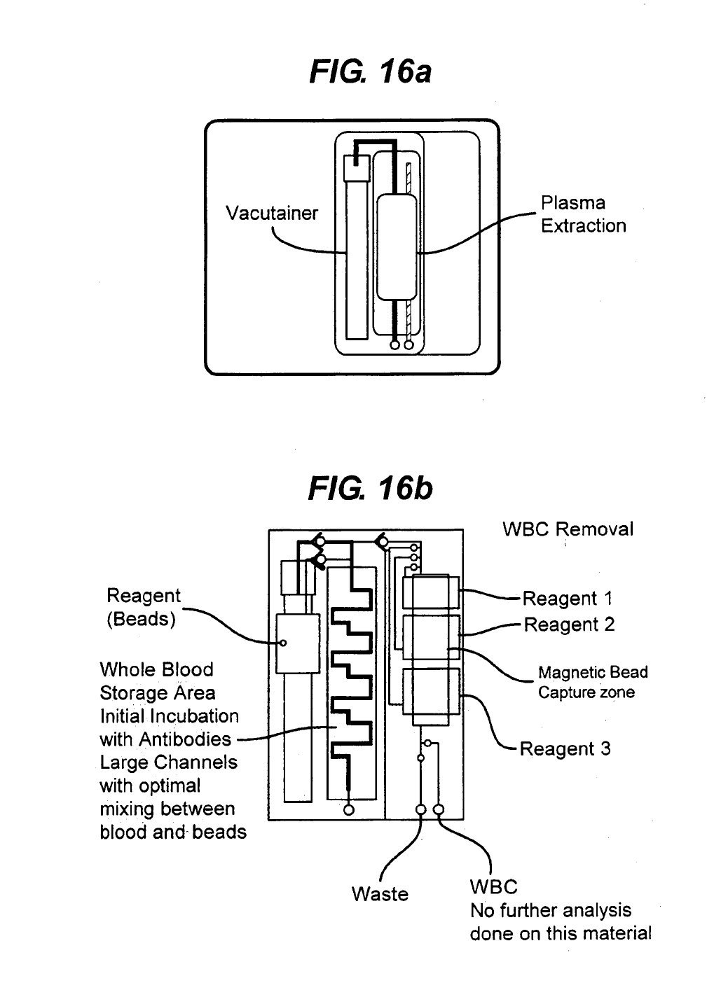

FIGS. 16a and 16b show example layouts for a sample preparation component that is intended to prepare sample from whole blood. FIG. 16a shows the sample zone of FIG. 12 (designed to accept a blood tube) with a layout for extracting plasma. The plasma is made ready to be employed in a further assay, for example an assay as set out in FIG. 14. FIG. 16b shows an exemplary layout for separating white blood cells form the sample.

### Example Layout for Processing Components for Specific Assays

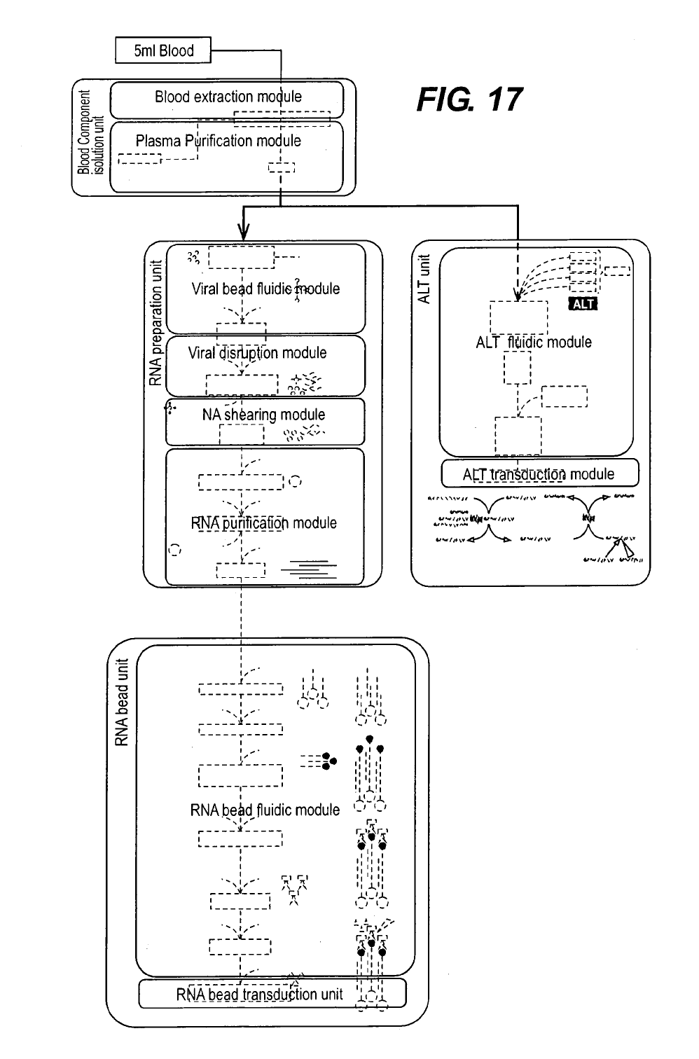

FIGS. 17-21 show processing component layouts for five specific assays;
17. An HCV monitoring chip comprising an HCV quantitative assay
18. An HCV (or HIV) bead chip comprising an HCV (or HIV) bead assay
19. An HCV surface chip comprising a viral screening assay for the genotype and serology
20. An HCV primary screening chip comprising an HCV genotyping assay and ALT assay
21. A highly multiplexed HCV monitoring assay

### Example Assay System Comprising a Cartridge of the Invention

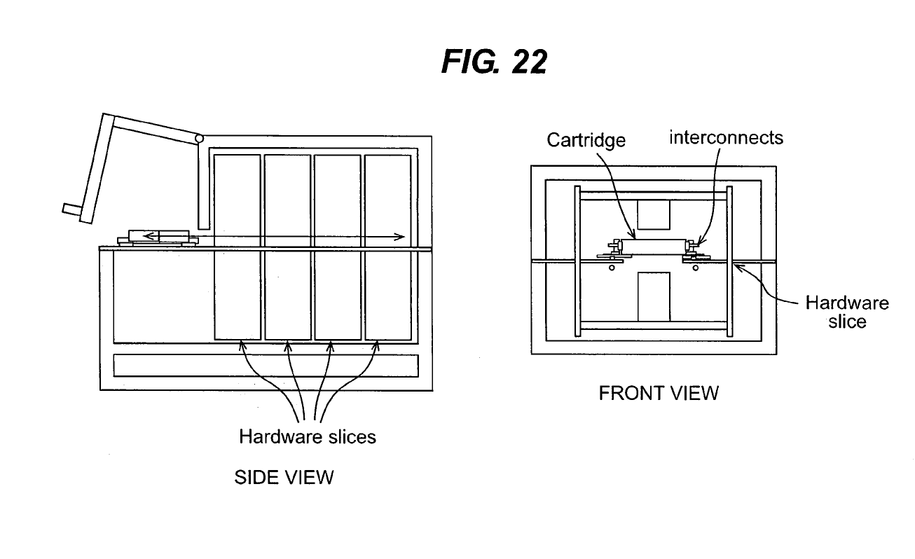

An example of the assay system of the present invention is depicted in FIG. 22. This figure shows a side view and front view of an assay device comprising the cartridge of the invention. The hardware slices and the cartridge and interconnects with the hardware slices are shown.

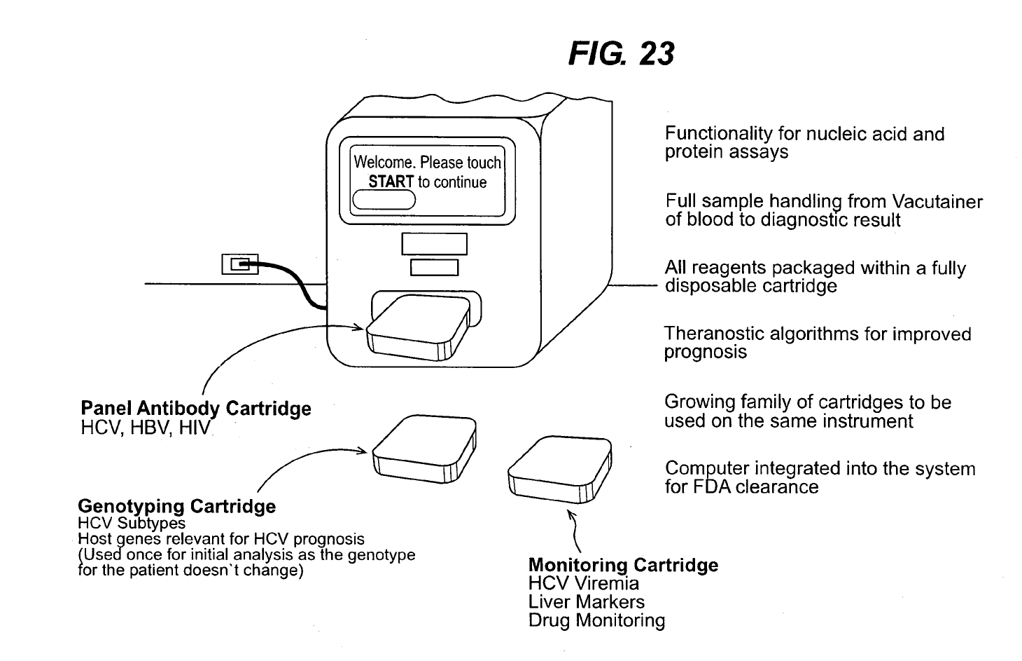

The whole assay system, depicting the assay device and several cartridges, and illustrating the near patient environment utility of the system is set out in FIG. 23.

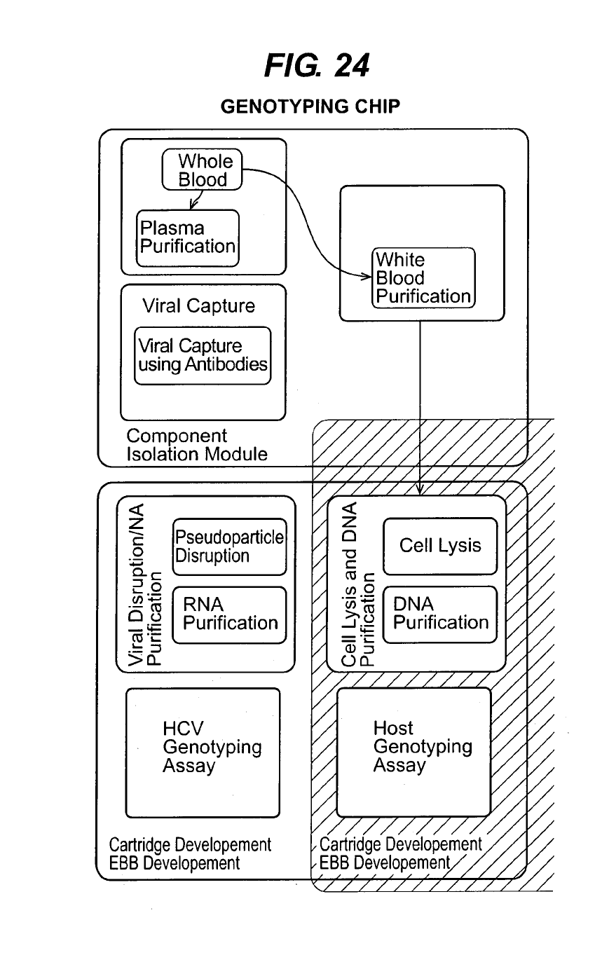

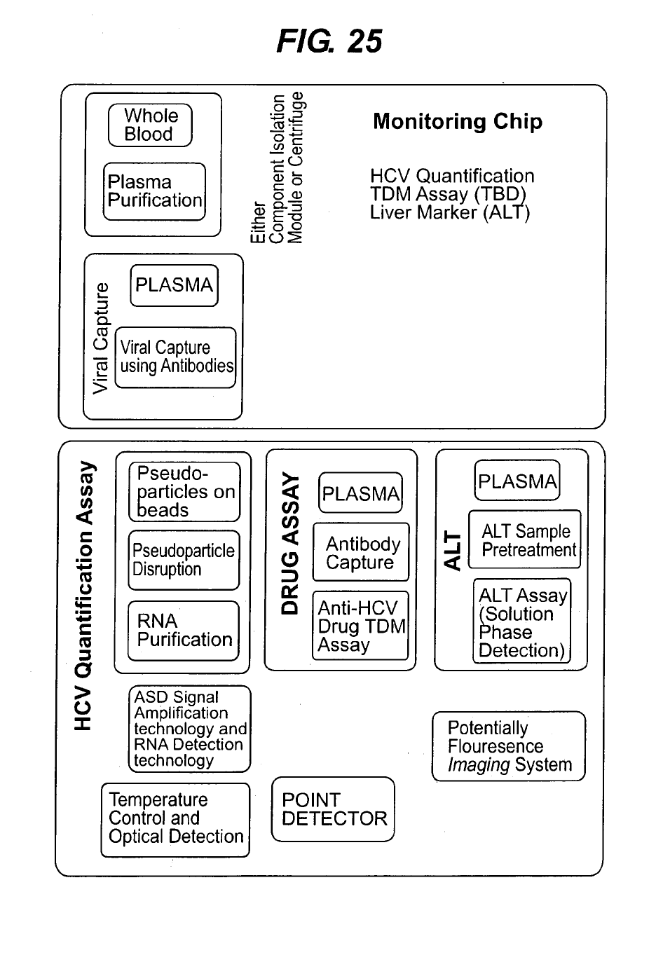

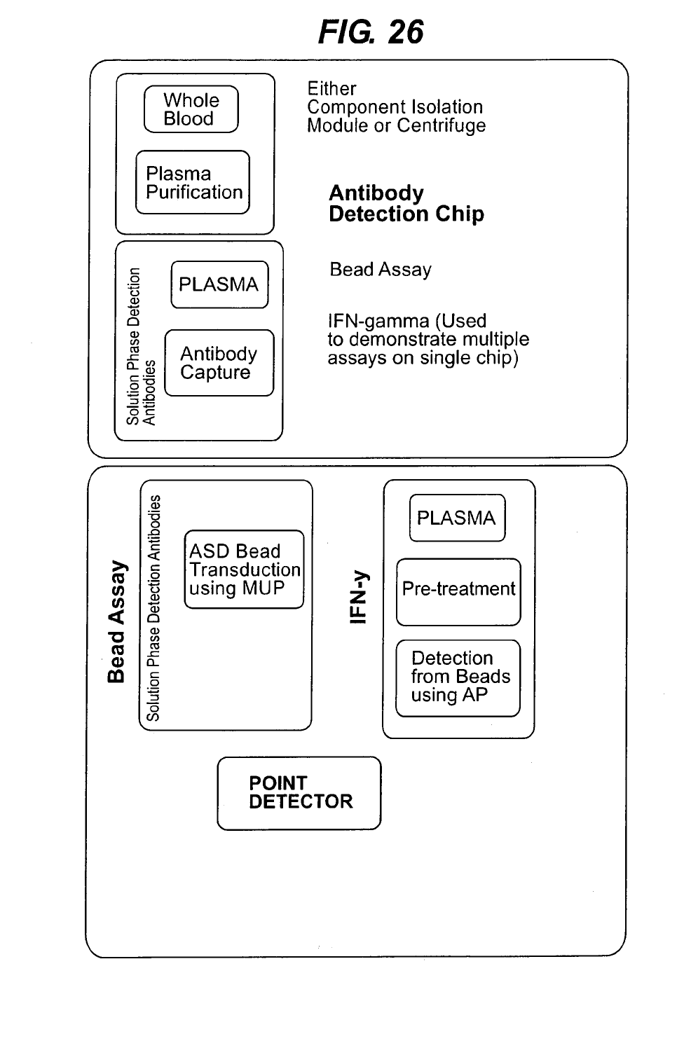

A more detailed illustration of the modules making up a genotyping cartridge, a monitoring cartridge and an antibody cartridge are shown in FIGS. 24, 25 and 26 respectively.

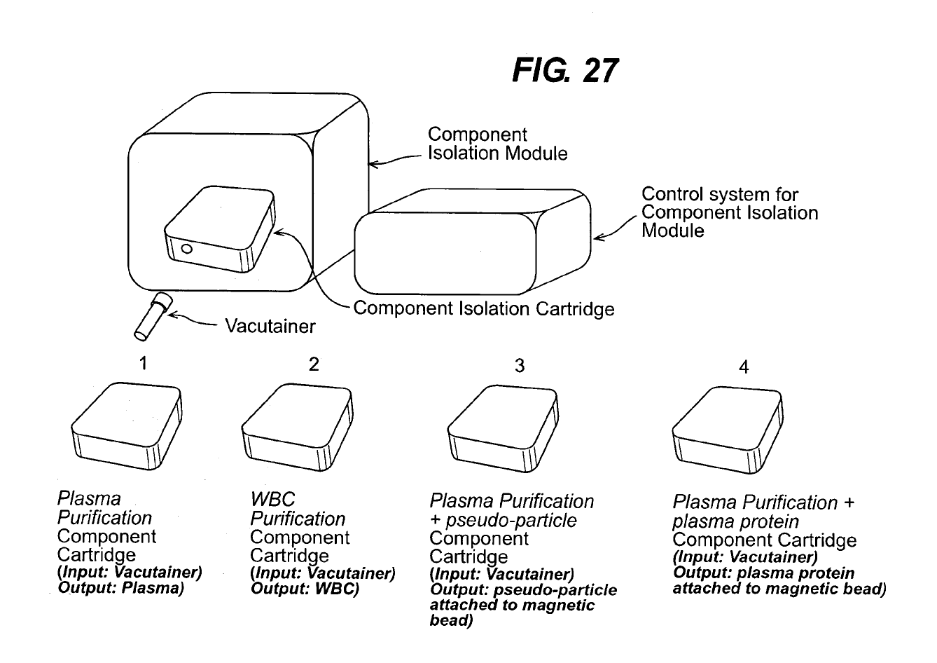

A more detailed illustration of sample preparation components is depicted in FIG. 27.

### Cartridge Interface to a Processing Instrument

The cartridge can be entered into a slot as in FIG. 1, alternatively it can be placed into a drawer and the drawer is then slid into the instrument.

The present invention will now be described in more detail. The cartridge system of the invention may comprise the following aspects: a reagent storage component; a reagent processing component; an optional sensor component; a further optional sample preparation component; a means for coupling the storage component to the processing component; a means for coupling the sample preparation component to the storage component and/or the reagent component; a means for liquid coupling between the reagent storage component and the reagent processing component; a means for liquid coupling the sample preparation component to the reagent storage component and/or the processing component. An example of how the cartridge may be interfaced to a processing instrument will be described, and an example of how such a cartridge can run a biological assay will also be discussed.

In the present invention, the height of the reaction chamber is not especially limited, but in preferred embodiments it is within the range 10 μm to 50μ. It is advantageous that the sensing element can be situated on the reagent cartridge, in which case the processing component may form the other side of the reaction chamber. In this embodiment, the method and configuration for coupling the two components may control the chamber height. When the chamber height is important to assay function, then a machine-mediated coupling is preferred (see below). The “top” of the chamber contained within the processing layer may be a transparent window, such that an optical sensor for viewing the sensor surface need not (conveniently and advantageously) require a viewing aperture in the reagent cartridge. Such an arrangement is also compatible with the sensor substrate incorporating a pattern of electrodes which may be used for electro-chemical detection.

Typically the sensor component is prepared with biological probes, which may be specific to the type of assay being carried out. These probes may, for example, be localised zones with surface attached antibodies (for protein capture) or oligomers (for nucleic acid capture). It is convenient that these probes, which will be assay specific, are attached to the reagent cartridge since this may also be assay-specific.

The type of sensing element for detecting an analyte is not especially limited. It may be selected depending on the assay method and/or analyte in question. Typically the element comprises one or more of a biosensor array, an electrochemical biosensor element, and an optical biosensor element.

In the present invention it is preferred that the reagent component and/or the processing component and/or the sensing component comprises one or more connection ports for establishing one or more connections with the other component(s), on coupling the components together. Preferably, the one or more connection ports are comprised of one or more inlet ports and/or one or more outlet ports. Typically, one or more or all of the connection ports of the reagent component and/or of the processing component and/or the sensing component comprise a seal to seal the internal contents of the component from the surroundings. Preferably one or more or all of the reagent component and the processing component and the sensing component comprise a connection means for facilitating coupling the components together, which connection means is configured to break the seal of a connection port so as to establish a sealed connection between the reagent component and the processing component on coupling. The connection means may, for example, take the form of a short needle, of the type employed in a syringe, which has a sharpened and tapered point for piercing a seal and penetrating the other component to ensure liquid is delivered into the other component by establishing a fluid connection. Preferably the seal, when pierced, forms a further seal around the connection means to ensure that the internal spaces of the cartridge remain isolated from the surroundings. This may be achieved by selecting appropriate material for the seal.

An example of such an embodiment of the invention is shown in FIGS. 11a and 11b. In this example, the reagent component (reagent cartridge in the Figure) is coupled to the processing component (microfluidic device in the Figure) using an elastomer interconnect comprising a needle. The needle sits in registry with the correct portions of each component (first diagram of FIG. 11b) and when pressure is applied to the two components to ‘snap’ them together, the reagent component is pierced forming a fluidic path into the processing cartridge. The inter-connect is typically made from moulding PDMS in a custom built tool. Alternatively it may be made from a thermoplastic elastomer and glued on to the microfluidic device. The inter-connect is designed such that the needle doesn't pierce the reagent cartridge until a leak-free seal has been generated between the reagent cartridge and the microfluidic device.

In a further preferred embodiment, the cartridge system is configured such that coupling the reagent component to the processing component causes one or more reagents to enter the processing component from the reagent component. Thus, coupling may be employed to initiate a “priming” cycle, e.g. by flooding the device with appropriate liquids, such as buffer solution. This may be achieved by including a ‘pumping’ means, which means is preferably driven by means of the coupling step. Such microfluidic pumping means are well known in the art.

The cartridge system of the present invention will typically be employed in a biological assay. In such assays, it is the norm to test a sample from a patient in order to establish a diagnosis (sometimes in combination with a preferred treatment—termed theranostics). Thus, in most embodiments the reagent component and/or the processing component and/or the sample preparation component comprises a sample zone, configured to accept a sample. The location of the sample zone is not especially limited, provided that it is suitable for the particular assay in question. Sometimes the sample zone is not present in the cartridge system at all, but is instead in the assay device. However, preferably the sample zone is in the processing component (and more preferably in the sample preparation component when present). In preferred embodiments, the sample zone is configured to deliver the sample to the processing component.

The sample will be assayed with a view to detecting the identity and/or quantity of a particular analyte which may be in the sample. The type of analyte is not especially limited, and the cartridge system of the invention may be adapted to many types of analyte, including assays for multiple analytes, sequentially or simultaneously. Typically, the analyte is selected from a biological molecule, a virus or virus component, and a cell or a cell component. Examples of analytes include whole cells such as liver cells, enzymes, whole viruses (e.g. Hepatitis C virus (HCV) and Human Immunodeficiency Virus (HIV)), proteins polypeptides and peptides, and nucleic acids such as DNA and/or RNA. Also included are carbohydrates and small molecules, such as drugs, pharmaceuticals and metabolites.

Typically, the processing component comprises one or more microfluidic processing elements, although it is not essential that the processing component is a microfluidic element to obtain all of the advantages of the system. Generally, the processing component comprises a plurality of processing zones. These are not especially limited, but are typically selected from one or more of an analyte and/or sample preparation zone, an analyte and/or sample separation zone, an analyte and/or sample concentration zone, an analyte and/or sample amplification zone, an analyte and/or sample purification zone, an analyte and/or sample labelling zone and an analyte and/or sample detection zone. Typically the reagent component comprises a plurality of reagent storage zones. These may comprise one or more reagents suitable for carrying out one or more processing steps selected from analyte and/or sample preparation, analyte and/or sample separation, analyte and/or sample concentration, analyte and/or sample amplification, analyte and/or sample purification, analyte and/or sample labelling, and analyte and/or sample detection.

As mentioned above, in some embodiments a sample preparation component is present. This is particularly preferable if sample preparation is a particular problem for the assay involved, or if several different samples are to be used in the same assay (e.g. a blood sample and a urine sample would need different sample preparation in order that the samples could be processed through the same assay cartridge). When the sample preparation component is present, it may comprise one or more of the following areas or zones: a sample preparation zone, a sample separation zone, a sample concentration zone, a sample amplification zone, a sample purification zone, a sample labelling zone, and/or a sample quality control zone.

The sample preparation component may be formed of a single component that may be configured to attach to either or both of the reagent storage component or the processing component. This single component may be pre-coupled to either of the other components, or may be coupled to them by the user, either by hand or by use of an assay device. In some embodiments, the sample processing component comprises two sub-components: a sample preparation reagent component and a sample preparation processing component. These components may function in a similar way to the two components of the main cartridge—the reagent component providing the reagents necessary for sample preparation whilst the processing component uses these reagents in conjunction with the sample itself to prepare the sample for introduction into the processing component of the main cartridge to perform the assay. The sub-components may be pre-coupled or may be configured to be coupled together by the user.

The analyte/sample may be delivered attached to magnetic (or other) beads as may one or more reagents from the reagent component. If magnetic beads are employed, the connection means for coupling the components together, and any appropriate conduits in the components are appropriately configured to allow beads to move to and from the required zones in the components.

The invention also provides a method of forming a cartridge, which method comprises coupling a reagent component and a processing component and optionally a sensing component and further optionally a sample preparation component of a cartridge system as defined above. In one embodiment of the invention, a simple operator action may couple the components together. However, it is preferred that such an operator action registers the components together for loading into a processing apparatus (e.g. an assay device), and the processing apparatus effects a second (machine activated) extension of the operator action to finally couple the loaded components. Typically, this final machine coupling action takes place after the sample has been automatically loaded such that the sample is sealed within the cartridge. In a further alternative, the components are loaded into the processing apparatus (assay device) and the apparatus effects the coupling itself. In this embodiment there is no need for the operator to register the components together initially and they may be independently loaded into the apparatus if desired. It is especially preferred to use machine coupling where there are a larger number of components; in the present invention there may be for example up to 5 components if a reagent storage component, processing component, sensor component and two sample preparation sub-components are all present.

The present invention extends to all of the possible component arrangements, including 2, 3, 4 or 5 component systems, provided that the 2 essential components (reagent storage and processing components) are present.

Thus, in one preferred method of operation, the following exemplary sequence may be followed:
(i) load sample in processing component;
(ii) bring reagent component into registration with the processing component and optionally the sensing component and further optionally the sample preparation component;
(iii) couple (or ‘snap’) the components together (preferably using the processing apparatus).

This approach is preferred because it greatly reduces any risk of manual misregistration of the components (an expensive mistake for the user) which, even for a user with good aptitude could be incurred by, say, snapping one end together first with an attendant risk of leakage. The arrangement also eliminates any errors which may result from manual loading of the cartridge. It also enables the sample to be automatically loaded before the components are brought together, such that when they are brought together, all reagents and chemicals are entirely contained within the cartridge.

Also provided by the invention is a cartridge comprising a reagent component of a cartridge system as defined above coupled to a processing component of a cartridge system as defined above and optionally coupled to a sensing component of a cartridge system as defined above and further optionally to a sample preparation component of a cartridge system as defined above.

The invention still further provides an assay system, comprising:
(a) a cartridge system or cartridge as defined above; and
(b) an assay device arranged to accept a cartridge as defined above.

Yet further provided is an assay method for one or more analytes in a sample, which method comprises:
(a) introducing the sample into a sample zone of a reagent component and/or the sample zone of a processing component and/or the sample zone of a sample preparation component, in a cartridge system as defined above;
(b) coupling the cartridge system to an assay device configured to accept the cartridge; and
(c) assaying for the one or more analytes using the assay device.

The method preferably further comprises a step of coupling the reagent component to the processing component and optionally to the sensing component and further optionally to the sample preparation component to form a cartridge. This step is not required if the cartridge system is provided in a pre-coupled state (any one or more of the various components can be pre-coupled, if desired). If any one or more of the components are not pre-coupled, then this coupling step is required. As mentioned above, the coupling may be manual (i.e. carried out by the user) or may be carried out by the assay device. In the latter case, it is typical that the user places the components that need to be coupled in registry with each other and then introduces these into the device. The action of introducing the components into the device may force the components together, causing them to ‘snap’ or lock together, depending on the specifics of the cartridge design.

The invention also provides a reagent component for storing one or more reagents, which reagent component is configured to be coupled together with a processing component and optionally with a sensing component and further optionally with a sample preparation component to form a cartridge, wherein the reagent component comprises at least one compartment configured to accept waste from the processing component, and wherein the reagent component is not configured to take part in processing the reagents in the assay, except to accept waste from the processing component. In a preferred embodiment, the reagent component comprises at least one sensing component comprising a sensing element for detecting an analyte.

### Assays and Component Layouts

The present invention is not limited in terms of the assays that may be carried out on the processing component. Accordingly, the assays may be for screening, purifying, identifying, capturing and/or quantifying any type of substance and in particular any type of biological substance. The type of biological substance may be a pathogen that causes infection (such as a virus, a bacterium, a fungal agent or the like) or may be a biological characteristic of the patient (such as gene profiling, protein profiling, disease and prognosis profiling or the like—these may include DNA, RNA, protein, polypeptide, peptide, and enzyme assays, for example) or may even be a biologically significant chemical (such as small molecules, metabolites, pharmaceuticals and drugs). It is especially preferred that the assay provides information on disease existence and progression. Significant diseases of interest with the invention include, but are not limited to hepatitis (A, B and C), HIV, HPV (human papilloma virus) and the like.

Preferably, the processing component is a microfluidic component, since this allows assays to be carried out speedily on a small quantity of sample, which is ideal in the near-patient environment. However, in some instances larger macro layouts may be preferred.

Where the processing is microfluidic or not, there are four types of particularly preferred assay:
1. Nucleic acid assays (such as DNA or RNA).
2. Enzyme assays (an ALT (Alanine Aminotransferase, a liver enzyme) assay is especially preferred in the context of hepatitis infection).
3. Protein assays (typically using antibodies for detection, e.g. on a microarray—preferred analytes of interest include hepatitis (A, B and/or C) and interferon gamma (IFN-γ).
4. Small molecule assays (such as pharmaceuticals or drugs—typical methods involve competition assays using antibodies). Therapeutic drug monitoring (TDM) is also an option.

In the present invention, there are typically a number of functional units that are required to perform an assay. These units include, but are not limited to the following:
1. A blood component isolation unit, which extracts blood from the patient vacutainer and processes it into plasma.
2. A white blood cell (WBC) unit, which takes whole blood and extracts white blood cells.
3. A protein bead unit, which provides the on-bead assay for captured plasma antigens.
4. An RNA preparation unit, which purifies RNA from material (e.g. HCV) captured on-bead.
5. A protein surface unit, which provides the surface assay for captured antibodies (or antigens).
6. An enzyme unit which provides an enzymatic assay (e.g. an ALT assay).
7. An RNA bead unit, which provides the on-bead RNA assay from purified RNA (e.g. HCV RNA).
8. An RNA surface unit, which provides the surface based assay from purified RNA (e.g. HCV RNA).

Each functional unit may be sub-divided into modules (or sub-units) that provide the functions necessary for an assay step. These modules are shown in detail in FIG. 14. Broadly speaking each module corresponds to a process which involves specific design, engineering and optimisation.

Several assay steps (modules) can form an assay process (unit) or a full chip (processing component). Examples of preferred constituent modules for each of the above exemplary units are as follows:
1. Blood component isolation unit:
   a) A blood extraction module, which takes whole blood from a vacutainer and flows it into the blood filter or the WBC unit.
   b) A plasma-purification module, which processes whole blood through a cross-flow filter.
2. White Blood Cell (WBC) unit:
   a. A WBC purification module, which takes whole blood and captures eosinophils using a bead based method.
3. Protein bead unit:
   a) A protein bead fluidic module, which captures using a bead based method antigens from plasma and prepares them for signal transduction.
   b) A protein bead transduction module, which collects and transmits the signal resulting from the captured plasma antigens to the software device.
4. RNA preparation unit:
   a) A viral bead fluidic module, which captures, using a bead based method, viral particles from isolated plasma.
   b) A viral disruption module, which releases RNA from the captured viral particles.
   c) A nucleic acid shearing module, which cuts the viral genome into manageable fragments (i.e. without any secondary structure).
   d) An RNA purification module, which purifies sheared RNA fragments from other impurities in the mix.
5. Protein surface unit:
   a) A protein surface fluidic module, which captures and prepares antibodies from plasma onto surface for signal transduction.
   b) A protein glass integration module, which gathers and transmits the signal generated from captured antibodies to the software device.
6. Enzyme unit:
   a) An enzyme fluidic module, which mixes the necessary reagents with plasma for the enzymatic assay (typically an ALT assay).
   b) An enzyme transduction module, which collects the fluorescent signal from the enzymatic assay and transmits it to the software device.
7. RNA bead unit:
   a) An RNA bead fluidic module, which captures, using a bead based method, RNA fragments (e.g. HCV RNA) from plasma and prepares them for transduction.
   b) An RNA bead transduction module, which collects and transmits the signal generated from captured RNA fragments (e.g. HCV RNA) to the software device.
8. RNA surface unit:
   a) An RNA surface fluidic module, which captures onto a glass surface RNA fragments (e.g. HCV RNA) and prepares them for signal transduction.
   b) An RNA glass integration module, which collects and transmits the signal generated from captured RNA fragments (e.g. HCV RNA) to the software device.

Some of these modules may be functionally very similar. For example, the manipulation of beads with captured proteins or captured nucleic acids (NAs) requires the same fluidic functionality. However, in some cases it is likely that the number of fluidic lines, plastics, reaction temperatures, chamber configurations or assay steps will be different between two assays; such that they are actually quite different. Accordingly, in some cases multiple assays can use the same module or unit layout, whilst in other cases multiple assays use multiple module and/or unit layouts.

A chip layout for a nucleic acid assay is depicted in FIG. 14. The layout comprises a number of units and modules linked together so as to enable the full assay. These include:
1. A viral bead fluidic module (the virus capture bead section in the Figure where the beads are mixed with the plasma in a mixing chamber). This captures (using a bead-based method) viral particles from isolated plasma.
2. A virus disruption module (the subsequent washing, lysis buffering and transferring section). This releases RNA from the captured viral particles.
3. An RNA shearing module (marked nucleic acid shearing in the Figure). This cuts the viral genome into manageable fragments (i.e. without any secondary structure).
4. An RNA purification module (the section including the introduction of the binding buffer and the non-specific nucleic acid capture beads into the mixing zone, followed by the introduction of the high salt wash and the elution buffer which enter the RNA purification chamber, followed by washing away the waste). This purifies sheared RNA fragments from other fragments in the mix.
5. An RNA bead fluidic module (the introduction of sequence specific capture beads to mix with the purified RNA in the sequence specific capture area, followed by washing, adding a second probe, introducing enzyme, a second wash, adding substrate and a further wash). This captures, using a bead-based method, RNA fragments from plasma and prepares them for transduction
6. An RNA bead transduction module (the marked transduction chamber in the Figure)

Typically, the processed substance is then detected in the transduction chamber by means of the sensor.

FIG. 15 provides a more general example and is formulated with an HCV assay in mind, although it may be applicable to other assays. This system is more complex than that highlighted above in FIG. 14 (and may encompass that above), potentially involving many assays (see FIGS. 17-21). It may be used for (inter alia) detecting HCV, genotyping the virus, and monitoring liver enzyme ALT in the patient. The Figure also shows the sample preparation component where plasma and/or white blood cells may be selected (either in an integrated cartridge or otherwise—see above). A variety of units and modules are depicted, which may be needed for the various assays. Not all assays need take place within a single cartridge, and (for example) the detecting/genotyping assay may in fact be carried out on a separate cartridge from the ALT monitoring, such as a cartridge depicted above in FIG. 14. In this example the following modules (sub-sections) of the processing component are shown:
1. A protein bead unit having a protein bead fluidic module and a protein bead transduction module
2. An RNA preparation unit having a viral bead fluidic module, a viral disruption module, an RNA shearing module and an RNA purification module
3. A protein surface unit having a protein surface fluidic module and a protein glass integration module
4. An ALT unit having an ALT fluidic module and an ALT transduction module.

The RNA preparation unit may feed into two further units:
5. An RNA bead unit comprising an RNA fluidic module and a RNA bead transduction module
6. An RNA surface unit comprising an RNA surface fluidic module and an RNA glass integration module

The blood extraction module and white blood cell purification modules discussed in relation to FIG. 15 are shown in more detail in FIGS. 16a and 16b. Both of these may be present on the same sample preparation component (as two different modules) if desired. There may also be a blood extraction module (see FIG. 15, where this is depicted). The blood extraction module takes whole blood from the vacutainer and delivers it to the blood filter or white blood cell processing unit. The plasma purification module processes whole blood through a cross-flow filter. The WBC module takes whole blood and captures eosinophils using a bead-based method.

FIGS. 17-21 show various more specific assays that may be performed using the units and modules depicted in FIG. 15:
17. An HCV monitoring chip comprising an HCV quantitative assay
18. An HCV (or HIV) bead chip comprising an HCV (or HIV) bead assay
19. An HCV surface chip comprising a viral screening assay for the genotype and serology
20. An HCV primary screening chip comprising an HCV genotyping assay and ALT assay
21. A highly multiplexed HCV monitoring assay

The HCV monitoring chip illustrated in FIG. 17 demonstrates a sample to answer theranostic test. By performing measuring HCV viraemia in parallel to a liver function assay, this test fulfils many of diagnostic and theranostic goals. In a point of care (POC) situation, this chip may readily monitor patient disease by measuring response to treatment (for example—is the drug regime giving the expected log drop in viraemia over time?) along with corresponding liver-damage (blood ALT levels).

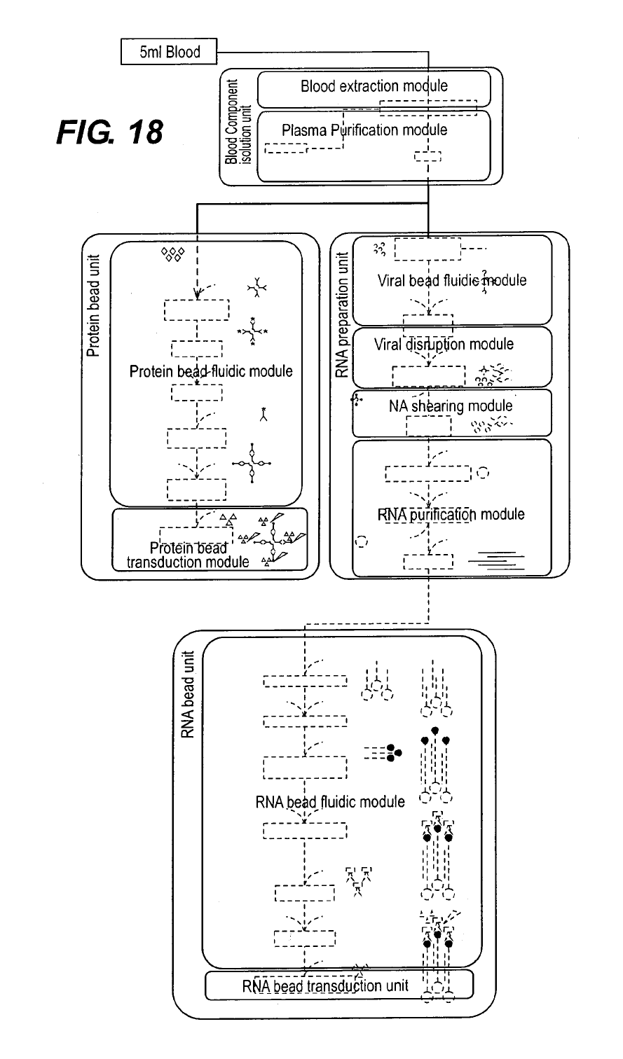

The HCV bead chip illustrated in FIG. 18 demonstrates full bead functionality. Using different, well chosen, protein targets for liver damage, this set-up may also provide a good monitoring device for liver disease progress.

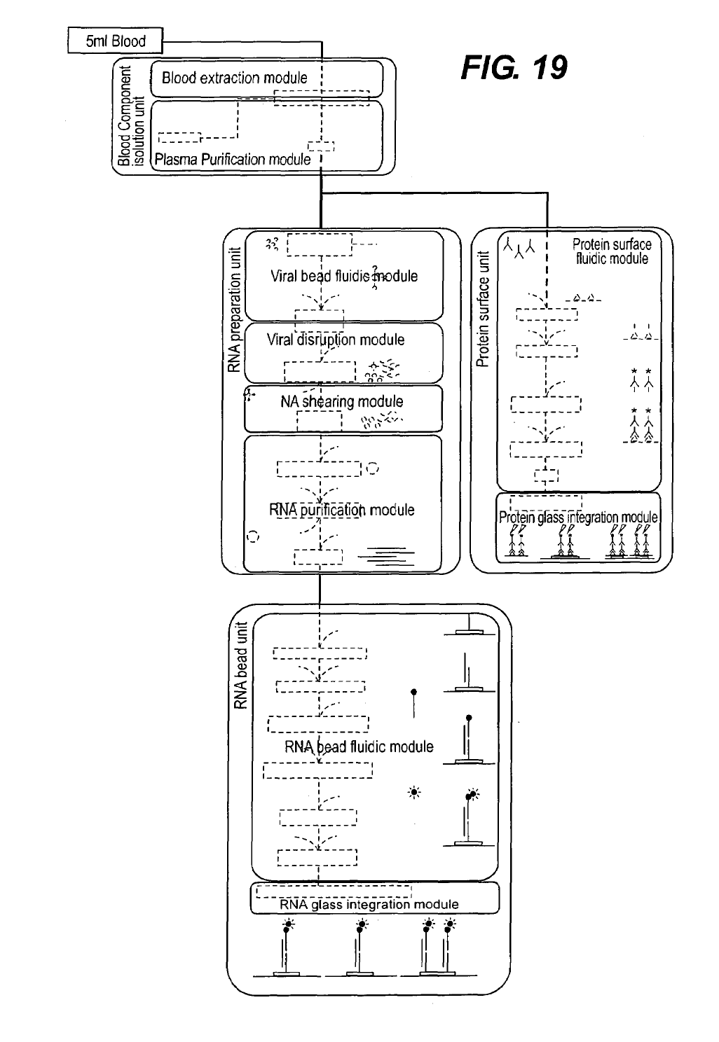

The HCV surface chip drawn in FIG. 19 demonstrates, thanks to its array capacity, a good viral screening device which searches for genotype and immunity to a panel of viruses and diseases. It could for example screen for all HCV, HIV and HPV viruses by determining patient exposure and current infection status (genotype and viraemia).

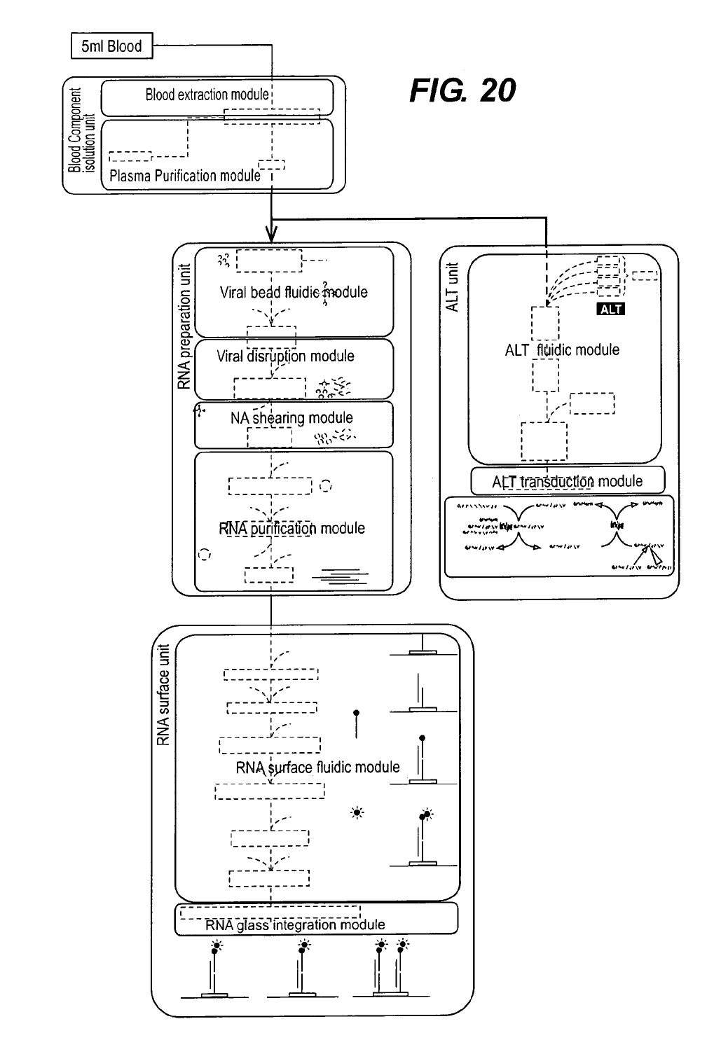

The HCV primary screen chip shown in FIG. 20 may be a primary screen test to determine HCV genotype and give an indication of the disease progress by incorporating the ALT assay. With HCV quantification it may also provide the same advantages as the HCV monitoring chip. Multiplexing of liver markers may also be advantageous here.

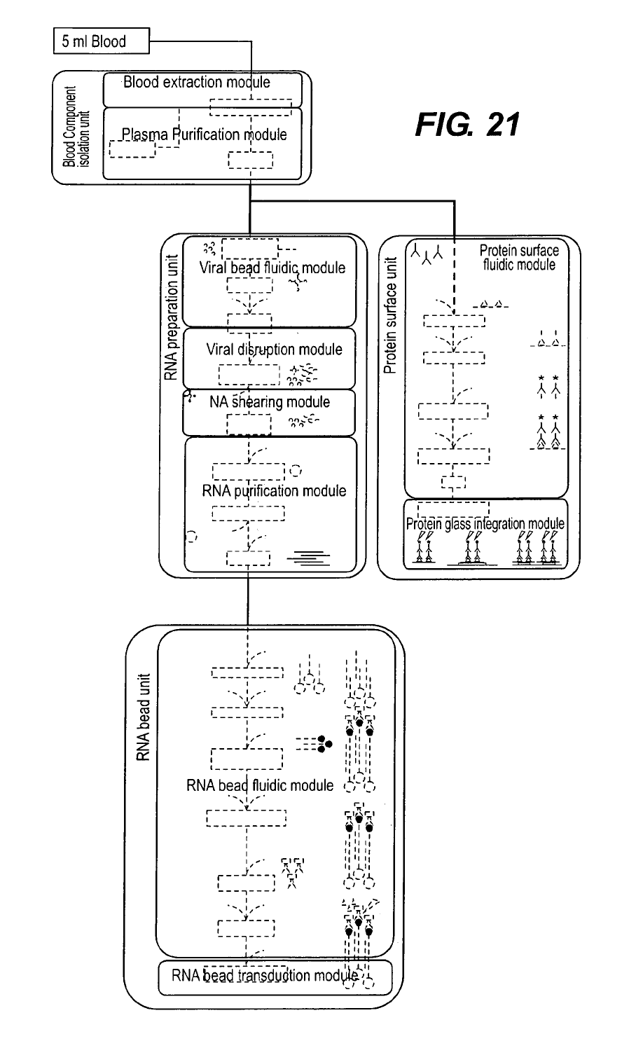

The Highly multiplexed HCV monitoring chip, shown in FIG. 21 provides an elegant solution to viral disease monitoring. As more protein biomarkers are being discovered, it is reasonable to think that viral disease monitoring will be more reliant on multiple indicators of disease progression. For a particular viral disease this chip measures the evolution of viraemia to low levels thanks to the speed and sensitivity of bead based methods, and monitors a potentially highly multiplexed panel of disease biomarkers.

### Hardware

In addition to the above components, an assay device, which makes use of the components and cartridges of the present invention, may also comprise further hardware units for aiding in the assay. Typically these units are termed hardware slices. The hardware slices are not especially limited, and may provide any further functionality, as desires, including:
1. Manipulation of magnetic beads within a component.
2. Fluorescence and luminescence detection from within a chamber of the processing and/or sensing component (or another component, e.g. for quality control of a sample).
3. Metering of fluids.
4. Heat control (to heat up a desired zone of a component).
5. Planar array transduction.
6. Ultrasonics (e.g. for virus disruption).
7. Electrical (connection of electrical lines for may purposes.
8. Software (for user control, and output of information, as well as data processing/algorithmic data analysis etc.).

An example of the assay system of the present invention is depicted in FIG. 22. This Figure shows a side view and front view of an assay device comprising the cartridge of the invention. The hardware slices and the cartridge and interconnects with the hardware slices are shown.

The whole assay system, depicting the assay device and several cartridges, and illustrating the near patient environment utility of the system is set out in FIG. 23. The cartridges shown are a panel antibody cartridge (e.g. for HCV, HBV, HIV and/or HPV), a genotyping cartridge (e.g. for HCV subtypes, and host genes relevant for HCV prognosis) and a monitoring cartridge (e.g. for HCV viraemia, liver markers and drug monitoring). Whilst the user waits, the system is capable of performing a number of assays (chosen according to the nature of the patient and the stage of treatment/disease) by employing the desired cartridge.

The genotyping cartridge is typically used in the early stages of treatment to assess the viral sub-type (1-6 in the case of HCV) and also to assess host genotypes that may influence how the patient responds to treatment and affect prognosis for recovery. The monitoring cartridge is designed for frequent testing to ascertain patient disease progression. Vireamia (virus quantification) is very useful in this respect as it indicates whether a patient is responding to treatment. Patient liver markers (such as ALT) are also desirable to monitor in the context of HCV, since they yield information on the degree of hepatitis. The cartridge may also incorporate monitoring of the concentration of drugs (e.g. HCV drugs) in the patient. This data provides the clinician with detail about metabolism and allows them to tailor the dose to the individual. The antibody cartridge may be a panel test for detecting antibodies in the patient sample. It may be used in an initial test to determine whether the patient is infected with various diseases.

A more detailed illustration of the modules making up the genotyping cartridge, the monitoring cartridge and the antibody cartridge are shown in FIGS. 24, 25 and 26 respectively. In the genotyping chip, a sample preparation module obtains virus from whole blood, and an assay module quantifies nucleic acid. In the monitoring chip, the sample preparation also involves obtaining virus from whole blood, whilst in the assay module there is a small molecule (drug) assay, an ALT assay and an HCV quantification assay. In the antibody chip, antibody capture may take place on the sample preparation module, whilst a bead assay and/or an IFN-γ test may be employed.

A more detailed illustration of sample preparation components is depicted in FIG. 27. In this example the sample preparation components are shown as separate cartridges, although in other embodiments these components may be coupled to reagent storage and/or processing components. The output of these sample preparation components is not especially limited, as has been explained before, but in these examples the outputs are plasma, white blood cells, pseudoparticles attached to magnetic beads and plasma proteins attached to magnetic beads.

### EXAMPLES

### An Exemplary Microfluidic Biological Assay

FIG. 10 shows a reagent flow sequence corresponding to that required for a simple ELISA type assay.

### Key:

R1=Reagent storage 1
C3=reaction Chamber 3
W2=Waste storage 2

FIG. 10.1: Optional: The device is liquid primed by transferring buffer reagent from R1 through to W4

FIG. 10.2: A sample of human serum from a patient with or without HCV infection is loaded into C1, which contains magnetic beads chemically coupled to antigens from HCV. The antigens could be for example epitopes NS 3, 4 and 5 of HCV core protein (Cp21). The components two are incubated for several minutes. This incubation time allows human antibodies to HCV (anti-HCV hIgGs), if found in the patient serum, to bind to the HCV antigens found on the beads.

FIG. 10.3: A magnetic field is applied to C1 to aggregate the beads in the chamber, and the liquid left over from the reaction is transferred to waste chamber W1. Wash solution from R1 is introduced into C1, and the magnetic field is released. The system is incubated for several seconds to allow the beads to disperse. This procedure is repeated 3 times.

FIG. 10.4: The beads in wash solution are transferred to C2. A magnetic field is applied to C2 to aggregate the beads in the chamber, and the liquid left over from the reaction is transferred to waste chamber W2.

FIG. 10.5: A solution containing antibodies raised to human IgGs (anti-hIgG) that are coupled with Horseradish Peroxidase (HRP) is introduced into chamber C2, and the magnetic field is released, to allow the magnetic beads to disperse. The mix is incubated for several minutes. This incubation time allows the anti-hIgGs to bind to the anti-HCV hIgGs that were potentially found in the human serum

FIG. 10.6: The contents of chamber C2 is transferred to C3. A magnetic field is applied to C3 to aggregate the beads in the chamber, and the liquid left over from the reaction is transferred to waste chamber W3.

FIG. 10.7: Wash solution from R3 is introduced into C3, and the magnetic field is released. The system is incubated for several seconds to allow the beads to disperse. This wash procedure is repeated 3 times.

FIG. 10.8: The beads in wash solution are transferred to C4. A magnetic field is applied to C4 to aggregate the beads in the chamber, and the liquid left over from the reaction is transferred to waste chamber W4.

FIG. 10.9: A solution containing a substrate for HRP, such as luminol in the presence of hydrogen peroxide (H₂O₂), is introduced into C4.

FIG. 10.10: The resulting chemiluminescent signal is monitored via the optical system, which has access via the window of the cartridge. The strength of this signal represents the quantity of anti-HCV hIgGs present in the patient sample.

1. A cartridge system comprising:
(a) a reagent component for storing one or more reagents; and
(b) a processing component for processing the one or more reagents in an assay;
wherein the reagent component and the processing component are configured to be coupled together to form a cartridge, and wherein the reagent component and/or the processing component comprise at least one compartment configured to accept waste from the assay, the reagent component not taking part in processing the reagents in the assay, except to accept waste from the processing component.

2. A cartridge system according to claim 1, further comprising a sensing component comprising at least one sensing element for detecting an analyte.

3. A cartridge system according to claim 1, wherein the reagent component or the processing component comprises the sensing component.

4. A cartridge system according to claim 3, wherein the sensing component is removably attached to the reagent component and/or the processing component.

5. A cartridge system comprising:
(a) a reagent component for storing one or more reagents;
(b) a processing component for processing one or more reagents in an assay; and
(c) a sensing component comprising at least one sensing element for detecting an analyte;
wherein the reagent component, the processing component and the sensing component are separate components configured to be coupled together to form a cartridge.

6. A cartridge system according to claim 5 wherein the sensing component is configured to be coupled, optionally removably coupled, to the reagent component prior to coupling with the processing component.

7. (canceled)

8. A cartridge system according to claim 5, wherein the reagent component and/or the processing component comprise at least one compartment configured to accept waste from the assay.

9. A cartridge system according to claim 1, further comprising a sample preparation component for preparing a sample for the assay.

10. A cartridge system comprising:
(a) a reagent component for storing one or more reagents;
(b) a processing component for processing one or more reagents in an assay; and
(c) a sample preparation component for preparing a sample for the assay;
wherein the reagent component and the processing component are configured to be coupled together to form a cartridge.

11. A cartridge system according to claim 10, wherein the sample preparation component is configured to be coupled together to the reagent component and/or the processing component to form a cartridge.

12. (canceled)

13. A cartridge system according to claim 10, wherein the sample preparation component is formed from two separate components, these being a sample preparation reagent component and a sample preparation processing component.

14. (canceled)

15. A cartridge system according to claim 10, further comprising a sensing component comprising at least one sensing element for detecting an analyte.

16. A cartridge system according to claim 15, wherein the reagent component or the processing component or the sample preparation component comprises the sensing component.

17. (canceled)

18. A cartridge system according to claim 10, wherein the reagent component or the processing component or the sample preparation component comprise at least one compartment configured to accept waste from the processing component.

19. (canceled)

20. (canceled)

21. (canceled)

22. (canceled)

23. (canceled)

24. (canceled)

25. (canceled)

26. A cartridge system according to claim 1, which system is configured such that coupling the reagent component to the processing component causes one or more reagents to enter the processing component from the reagent component.

27. A cartridge system according to claim 10, wherein either the reagent component and/or the processing component comprises a sample zone, configured to accept a sample.

28. A cartridge system according to claim 27, wherein the sample zone is configured to deliver the sample to the processing component.

29. (canceled)

30. (canceled)

31. (canceled)

32. (canceled)

33. (canceled)

34. A cartridge system according to claim 2, wherein the sensing element for detecting an analyte comprises one or more of a biosensor array, an electrochemical biosensor element, and an optical biosensor element.

35. A cartridge system according to claim 2, wherein the analyte is selected from a biological molecule, a virus or virus component, and a cell or a cell component.

36. A cartridge system according to claim 35, wherein the analyte comprises DNA, RNA, a protein, a polypeptide, an enzyme, a carbohydrate, a pharmaceutical and/or a metabolite.

37. (canceled)

38. A cartridge comprising a reagent component of a cartridge system for storing one or more reagents coupled to a processing component of a cartridge system for processing the one or more reagents in an assay and optionally coupled to a sensing component and further optionally coupled to a sample preparation component.

39. An assay system, comprising:
(a) the cartridge as defined in claim 38; and
(b) an assay device arranged to accept the cartridge.

40. An assay method for one or more analytes in a sample, which method comprises:
(a) introducing the sample into a sample zone of the reagent component and/or the processing component and/or a sample preparation component, in a cartridge system as defined in claim 1;
(b) coupling the cartridge system to an assay device configured to accept the cartridge; and
(c) assaying for the one or more analytes using the assay device.

41. (canceled)

42. A reagent component for storing one or more reagents, which the reagent component is configured to be coupled together with a processing component and optionally with a sensing component and further optionally with a sample preparation component to form a cartridge, wherein the reagent component comprises at least one compartment configured to accept waste from the processing component, and wherein the reagent component is not configured to take part in processing the reagents in the assay, except to accept waste from the processing component.

43. A reagent component for storing one or more of the reagents, according to claim 42, wherein the reagent component comprises at least one sensing component comprising a sensing element for detecting an analyte.

44. An assay system comprising:
(a) an assay device component on which the assay takes place; and
(b) a hardware component comprising means for controlling and/or addressing the assay device;
wherein the hardware component comprises a plurality of separate modules, each module capable of a different controlling and or addressing function in respect of the assay device.

45. An assay system according to claim 44, wherein one or more of the modules are in the form of slices which are independently removable from the system, in order to provide the assay device with variable function.

46. An assay system according to claim 44, wherein the assay device comprises a cartridge system, cartridge, assay system and/or reagent component as defined in claim 1.

47. (canceled)

48. The assay method according to claim 40, wherein the sample is a whole blood sample, or a urine sample.

49. The assay method according to claim 40 wherein the sample is a mammalian sample.

50. The assay method according to claim 40 wherein the sample is a human sample.
